(19) United States
(12) Patent Application Publication
(10) Pub. No.: US 2004/0235857 A1
(43) Pub. Date: Nov. 25, 2004

(54) Crystalline therapeutic agent

(75) Inventors: Christopher Ian Dallman, Sandwich (GB); Andrew Kevern Hills, Sandwich (GB); James Gerard McGovern, Sandwich (GB); Richard Anthony Storey, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/763,794

(22) Filed: Jan. 22, 2004

Related U.S. Application Data
(60) Provisional application No. 60/449,185, filed on Feb. 21, 2003.
(60) Provisional application No. 60/508,051, filed on Oct. 1, 2003.

(30) Foreign Application Priority Data
Feb. 11, 2003 (GB) 0303097.0
Sep. 11, 2003 (GB) 0321309.7

(51) Int. Cl. A61K 31/519; C07D 487/02

(52) U.S. Cl. 514/252.16; 544/262

(57) ABSTRACT

A novel solid form of sildenafil citrate wherein the ratio of sildenafil: citrate is 1:0.5.

The present invention relates to a novel solid form of sildenafil citrate.

Anhydrous sildenafil mono-citrate has formula (I):

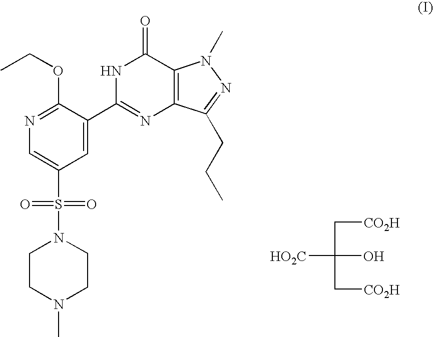

In particular the present invention provides a novel hydrated form of sildenafil citrate and processes for its preparation. The present invention additionally provides processes for the preparation of yet further hydrated and non-hydrated (de-hydrated) solid forms of sildenafil citrate via said novel hydrated form of sildenafil citrate.

Sildenafil is an orally active, potent and selective inhibitor of cyclic guanosine monophosphate (cGMP) specific phosphodiesterase type 5 (PDE5), which is the predominant PDE5 isoenzyme in human corpora cavernosa.

Sildenafil, 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one also known as 1-[[3-(6,7-di hydro-1-methyl-7-oxo-3-propyl-1H-pyrazolo[4,3-d]pyrimidin-5-yl)-4-ethoxyphenyl]sulphonyl]-4-methylpiperazine can be prepared according to the processes disclosed in EP0463756, WO94/28902, EP0812845, and EP0994115.

Sildenafil mono-citrate is a commercial compound marketed as Viagra® for the treatment of male erectile dysfunction. The commercial salt is the mono citrate salt, also known as anhydrous sildenafil citrate and has a molar ratio of sildenafil:citrate of 1:1.

Sildenafil monocitrate can be prepared from sildenafil free-base via reaction with citric acid via techniques such as are known to the skilled chemist and as exemplified hereinafter.

For successful utility within the pharmaceutical industry it is critical that the physicochemical properties of an active material are either known or can be reasonably predicted throughout the necessary processes during both its manufacture and pharmaceutical processing as well as during its shipping, storage and eventual therapeutic use. In some cases compounds can exhibit desirable medicinal properties, which cannot be translated directly into a suitable pharmaceutical composition, because the active compound itself has unsatisfactory physical properties such as for example poor chemical or processing properties.

The present invention provides new crystalline solid forms of sildenafil hemi-citrate, specifically novel hydrated forms of sildenafil hemi-citrate and in particular a lower hydrated form of sildenafil hemi-citrate. The novel hydrated solid forms, and especially the lower hydrated form according to the present invention has good physiochemical properties; desirable stability characteristics; desirable medicinal properties, good processing properties. The novel solid forms, and especially the lower hydrated form of the present invention can be incorporated into a variety of different formulation vehicles making it especially suited for pharmaceutical utility.

Alternative structural forms of sildenafil citrate (polymorphs) have been proposed in “Solid Modifications in Sildenafil Citrate”, Isra M. Admour, Muraz Sh. Salem, Naji Najib and A. A. Badwan (Proc. 3^{rd} World Meeting APV/APGI, Berlin, 3/6 April 2000, pages 639-640). No hydrated forms of sildenafil citrate have been reported.

According to one embodiment, the present invention provides novel solid forms of sildenafil citrate wherein the molar ratio of sildenafil:citrate is about 1:0.5. These 1:0.5 forms of sildenafil citrate are referred to hereinafter as sildenafil hemi-citrates.

According to a preferred embodiment said sildenafil hemi-citrate is present in a hydrated form. The present invention provides two hydrated forms of sildenafil hemi-citrate, a lower hydrated form and a higher hydrated form. The present invention additionally provides a dehydrated form of sildenafil hemi-citrate. This dehydrated form is referred to hereinafter as sildenafil hemi-citrate dehydrated hydrate.

The lower hydrated form of sildenafil hemi-citrate can be prepared and isolated according to the process described hereinafter. During the process for the preparation of the lower hydrated form of sildenafil hemi-citrate, a second higher hydrated form is produced.

Thus the present invention additionally provides processes for the preparation of two hydrated forms of sildenafil hemi-citrate.

Thus according to a further embodiment the present invention provides a lower hydrated solid form of sildenafil hemi-citrate having a powder X-Ray diffraction (PXRD) pattern substantially as defined hereinafter.

Thus according to a further embodiment the present invention provides a higher hydrated solid form of sildenafil hemi-citrate having a powder X-Ray diffraction (PXRD) pattern substantially as defined hereinafter.

In addition to potential formulation benefits this new, stable, crystalline lower hydrated form of sildenafil hemi-citrate is highly desirable as crystalline materials are, in general, more stable than their amorphous counterparts, they have a finite structure which can be reproducibly characterised by powder X-ray diffraction (PXRD) which can be used to identify the presence of a specific crystalline polymorphic form.

Sildenafil Hemi-Citrate Lower Hydrate

According to a preferred embodiment the present invention provides a lower hydrated solid form of sildenafil hemi-citrate. This material can be characterised by its powder X-Ray diffraction pattern (PXRD). ¹H NMR can be used to characterise the relative ratios of sildenafil to citrate within the lower hydrated form.

Preliminary dynamic vapour sorption (DVS) studies indicate that the amount of water present within the lower crystalline hydrated material can be expressed as a percentage wherein the amount of water present is between about 4% and about 7% and is preferably between about 5% and about 6% and is more preferably about 5.5% of the dry weight of the solid when equilibrated at 25% to 40% RH. The stoichiometry of water to salt within this lower crystalline hydrated material is about 1.5:1 to about 2:1 and is approximately about 1.7:1. From a nomenclature viewpoint this form may also be conveniently referred to herein as a “di hydrate” of sildenafil hemi-citrate. Vapour sorption data indicates water is bound within the crystal lattice.

As will be understood by the skilled solid-state chemist the relative amount of water will be dependant upon the crystallinity and/or purity of the sample as well as the specific conditions of analysis, such as the relative humidity (% RH).

Thus, according to a further aspect the present invention provides a novel lower hydrated solid form of sildenafil hemi-citrate, which at ambient conditions of temperature and humidity, accommodates approximately two water molecules per unit cell. Ambient conditions of temperature and humidity as defined herein means temperatures of between about 20 to about 30° C. and relative humidity (% RH) of about 25 to 50%. Preferred conditions herein are temperatures of about 30° C. and relative humidity (% RH) of about 30 to 45% RH.

As will be understood by the skilled solid-state chemist it may be possible to make and analyse the lower hydrated form of sildenafil hemi-citrate and/or mixtures thereof with other forms at temperatures and relative humidities outside of these ranges.

According to a preferred aspect the present invention provides a lower hydrated form of sildenafil hemi-citrate which contains between 5 and 6% water at from about 30 to about 45% RH and at about 30° C.

The preparation of the lower hydrated form of sildenafil hemi-citrate according to the present invention may be carried out as illustrated in Example 2 and in the Preparations sections hereinafter.

The lower hydrated form of sildenafil hemi-citrate according to the present invention can be prepared either from sildenafil mono-citrate (1:1) or from sildenafil free-base. Whilst this lower hydrated material can be formed from slurries of sildenafil mono citrate and an appropriate buffer across a broad pH range (from about pH 4 up to about pH 9.7) selection of non-optimal conditions can lead to the preparation of mixtures of hydrated material with sildenafil mono-citrate and/or sildenafil free-base.

According to a further embodiment the present invention provides a process for the preparation of the lower hydrated solid form of sildenafil hemi-citrate, starting from sildenafil mono-citrate via:

(i) treatment of the sildenafil mono-citrate with a solution of an acidic buffer;

(ii) isolation of solids;

(iii) vacuum drying at ambient conditions for up to 12 hours or conventional drying at 50 to 80° C. for between about 12 and 24 hours; and

(iv) re-exposure of the dried solids to ambient conditions.

The reaction (stage (i)) can be carried out from about 8 hours to in excess of 72 hours, preferably from about 8 to about 36, more preferably from about 8 to about 24 hours and especially from about 8 to about 12 hours.

Thus according to a preferred embodiment the present invention additionally provides a process for the preparation of the lower hydrated solid form of sildenafil hemi-citrate, starting from sildenafil mono-citrate via:

(i) treatment of the sildenafil mono-citrate with a solution of an acidic buffer at a pH in the range of from about 4 to about 7.5;

(ii) isolation of solids at about the temperature of the stage (i) reaction, preferably from 0 to about 25° C.;

(iii) vacuum drying at ambient conditions for up to 12 hours; and

(iv) re-exposure of the dried solids at about 25° C. and about 40% RH for up to about 24 hours.

According to a further preferred embodiment the present invention additionally provides a process for the preparation of the lower hydrated solid form of sildenafil citrate via treatment of mono-citrate, preferably as a slurry, with citrate buffer at a pH of about 6.4, a temperature of about 4° C., and a concentration of about 62.5 mg/mL, for up to about 24 hours, preferably for between about 8 and about 12 hours, followed by solid isolation via filtration at a temperature of from 0 to about 25° C., preferably at about 2 to about 10° C., more preferably at about 4° C., with subsequent drying under vacuum at room temperature for about 12 hours and then re-exposure of the dried solids to ambient conditions for up to about 24 hours.

According to a yet further embodiment the present invention additionally provides the product obtainable by the aforementioned processes.

For conversion of sildenafil mono-citrate to the lower hydrated form of sildenafil hemi-citrate any acidic buffer can be used which is capable of delivering the requisite pH range. Suitable methods for the preparation of buffers for use herein are described in the Examples section hereinafter. The skilled chemist may utilise alternative methodologies as are known in the art for the preparation of acid/base buffers having the desired pH range. Suitable pH ranges for use herein include buffered solutions having a pH ideally greater than about 4 and less than about 9.8, preferably less than about pH 8, more preferably between about 4 to about 7.5, even more preferably between about 5 and 7.5, yet more preferably between about 6.0 and about 6.8, and especially between about 6.2 and about 6.6, and about pH 6.4 in particular. Such treatment with acid/base buffer in this pH range comprises mixing the acid/base buffer solution and sildenafil mono-citrate together at the desired pH.

Whilst any buffer capable of delivering a pH solution in the range of from about 4 to less than about 9.8 may be used for the conversion of sildenafil mono-citrate to the lower hydrated form of sildenafil hemi-citrate, preferred herein are acid/base buffers produced from acetic, citric or phosphoric acid. Most preferred as buffering agents herein is a pH 6.4 solution using 0.2M citric acid with sodium hydroxide. Other buffers include pH 7.2 solutions using 0.2M phosphoric acid with sodium hydroxide buffer.

The reaction can be carried out at from about 0° C. to about room temperature, preferably at from about 0 to about 15° C., more preferably from about 0 to about 10° C., more preferably still at from about 2 to about 8° C. and especially at about 4° C. Any suitable starting concentration of sildenafil mono-citrate in the buffer solution can be used provided that a slurry is achieved.

Preferred herein are slurries, which can be agitated, shaken or stirred using regular methods or equipment such as a magnetic stirrer flea, a mechanical stirrer rod, manual shaking or a rolling bed mixer. Especially preferred herein are slurries in the range of from about 50 mg/ml to about 125 mg/ml and most especially about 62.5 mg/ml slurry of sildenafil mono-citrate in a buffered solution. The timing of the reaction will depend upon the pH of the buffer solution, the temperature of the reaction and the concentration and particle size of the sildenafil mono-citrate. Whilst it is possible to treat sildenafil mono-citrate having a range of particle sizes, preferred particle sizes for sildenafil mono-citrate for use herein are between about 5 and 500 μm, preferred particle sizes used herein are between about 100 and 400 μm. Ideal temperature ranges would be between the freezing point of the slurry (dependant on the buffer system used) and ambient temperature. As detailed herein before preferred temperatures for use herein are temperatures of from about 0 to about 15° C., more preferably from about 0 to about 10° C., more preferably still at from about 2 to about 8° C. and especially at about 4° C. Highly preferred herein is the preparation of the novel lower hydrated form of sildenafil hemi-citrate at from about 2° C. to about 6° C. in a buffered solution at pH from about 6.2 to about 6.6, more preferably about pH 6.4, wherein the buffer is a citric acid/base buffer.

The reaction can be carried out from about 8 hours to in excess of 72 hours, preferably from about 8 to about 36, more preferably from about 8 to about 24 hours and especially from about 8 to about 12 hours.

As discussed hereinbefore, in stage (ii), the resultant solids are isolated via filtration techniques at about the temperature of the aforementioned reaction, such as for example within about 5° C. or so. Preferred for use herein are vacuum filtration techniques.

In stage (iii), the filtered solid(s) are dried. Suitable drying techniques include vacuum drying at ambient temperature (from about 25 to about 30° C.) and at pressures of about 10 mBar, or conventional drying (oven-drying) at from about 50 to about 80° C. and at ambient pressure. Preferred for use herein is vacuum drying at room temperature for about 12 hours.

The desired lower hydrated form of sildenafil hemi-citrate is furnished from said filtered, dried solid material (the material from stage (iii)) following exposure to ambient conditions of temperature and humidity (about 25° C. and about 40% RH) for up to about 24 hours.

Sildenafil Hemi-Citrate Higher Hydrate

In the aforementioned process a further hydrated form of sildenafil hemi-citrate is produced during the initial buffering stage, stage (i). Analysis of the residues from stage (ii) provides this further hydrated form of sildenafil hemi-citrate.

The conditions for preparation of this higher hydrated form of sildenafil hemi-citrate are detailed hereinbefore at stages (i) and (ii). Whilst preferred conditions for stage (i) are detailed hereinbefore the skilled chemist will appreciate that using alternative conditions of temperature, concentration and/or pH etc. can increase the possibility of forming mixed products, such as for example mixtures of the higher hydrated form of sildenafil hemi-citrate with sildenafil free-base and/or anhydrous sildenafil (I:I) mono citrate.

According to a preferred embodiment the present invention provides a higher hydrated solid form of sildenafil hemi-citrate. This material can be characterised by its powder X-Ray diffraction pattern (PXRD). ¹H NMR can be used to characterise the relative ratios of sildenafil to citrate within the higher hydrated form.

Preliminary dynamic vapour sorption (DVS) studies indicate that the amount of water present within the crystalline higher hydrated material can be expressed as a percentage wherein the amount of water present is between about 11% and about 15% and is preferably between about 12% and about 14% and is more preferably about 13%. The stoichiometry of water to salt within this crystalline higher hydrated material is between about 3:1 and about 6:1 and is approximately about 4:1. From a nomenclature viewpoint this form may also be conveniently referred to herein as a “tetra hydrate” of sildenafil hemi-citrate. Vapour sorption data indicates that water is bound within the crystal lattice.

Thus, according to a further aspect the present invention provides a higher hydrated solid form of sildenafil hemi-citrate. According to a preferred aspect the present invention provides a higher hydrated form of sildenafil hemi-citrate which contains between about 12% and about 14% water at >45% RH, preferably at from about 55% to about 70% RH, and at about 30° C.

As described hereinbefore, and as exemplified at Example 2 the lower hydrated form of sildenafil hemi-citrate according to the present invention can be produced from the material described herein as the higher hydrated form of sildenafil hemi-citrate.

According to a further embodiment the present invention provides a process for the preparation of the higher hydrated form of sildenafil hemi-citrate, starting from sildenafil mono-citrate via:

(i) treatment of the sildenafil mono-citrate with a solution of an acidic buffer;

(ii) isolation of solids; and

(iii) drying at ambient temperature and >45% RH.

Once formed, the higher hydrated form of sildenafil hemi-citrate can be obtained directly from the buffered solution (of stage (i) as described hereinbefore) via any conventional isolation technique e.g. filtration (stage (ii)), followed by drying of the resultant isolated solid residue (filtered solid) at ambient temperatures and a relative humidity of >45%. Suitable filtration methods and techniques are as described hereinbefore. Especially preferred herein are a reaction (stage (i)) and filtration (stage (ii)) temperature of between 0 to about 25° C., preferably at about 2 to about 10° C., more preferably at about 4° C. Once isolated the filtered solid, from stage (ii), is dried to furnish the desired higher hydrated form of sildenafil hemi-citrate. As detailed hereinbefore suitable drying conditions for said filtered solid are ambient temperature and >45% RH and preferably 55% to about 70% RH.

According to a preferred embodiment the present invention additionally provides a preferred process for the preparation of the higher hydrated form of sildenafil hemi-citrate, as defined herein, starting from sildenafil mono-citrate via:

(i) treatment of the sildenafil mono-citrate with a solution of an acidic buffer at a pH in the range of from about 4 to about 7.5 at a temperature in the range of from about 0 to about 25° C.;

(ii) isolation of solids at about the temperature of the stage (i) reaction, preferably from 0 to about 25° C., and at a relative humidity of >45% RH;

(iii) drying at ambient temperature and from about 55% to about 70% RH.

According to a highly preferred process herein the higher hydrated form is isolated from the buffered solution via filtration, at a constant temperature of between about 0 to about 25° C., preferably at about 2 to about 10° C., more preferably at about 4° C.

According to a yet further embodiment the present invention provides a highly preferred process wherein the higher hydrated form is isolated from the buffered solution via filtration, at a constant temperature of between about 0 to about 10° C., preferably from about 2° C. to about 8° C., and especially about 4° C. and subsequently dried at about ambient temperatures and at an RH of between about 55% and 70%.

The present invention additionally provides the product obtainable by the abovementioned processes.

The hydrated forms of sildenafil hemi-citrate, the lower hydrate and the higher hydrate, according to the present invention can be characterised using a variety of analytical techniques. Powder X-Ray Diffraction (PXRD) was used to assess the structure of the solid forms. Thermogravimetric analysis (TGA) was used to qualify the relative weight loss when a sample of a particular solid form was heated from room temperature to about 120° C.

According to an alternative preparation the sildenafil hemi-citrate higher hydrate according to the present invention can be prepared by treating a mixture of sildenafil free-base and citric acid (anhydrous or monohydrated citric acid) at a range of high water activities in an organic solvent. The reaction can be carried out using a relative ratio of citric acid sildenafil free base of about 0.5:1 to about 0.75:1, preferably about 0.6:1.

Preferred for use in this alternative process are an acid:base ratio of about 0.6:1, in pure water, at a temperature of about 4° C. and for a time of up to 24 hours. Preferred isolation methods are filtration (as detailed hereinbefore in stage (ii)) at a temperature of about 4° C. Preferably the reaction is carried out for up to about 24 hours, more preferably from 12 to about 24 hours and especially about 12 to 15 hours.

High water activity as defined herein means 0.9 and above and is defined as the ratio of what the vapour pressure of water would be in the organic solvent/water mixture compared to pure water at that temperature. Preferred is a water activity of 0.95 and above, highly preferred is from about 0.98 to about 1.0 and especially preferred herein is pure water (activity of about 1.0). Suitable solvent/water mixture are reported in Zhu, H., Yuen, C., Grant, D. J. W., Int. J. Pharm. 135(1996), pages 151 to 160 the contents of which are included herein by reference. Preferred for use herein are solvent/water ratios of methanol/water in a volume (%) ratio of from at least about 10/90, more preferably about 5/95.

Highly preferred herein as solvent is methanol, alternative solvents such as ethanol, acetone or any other organic solvent can be used. Temperature range is from about 0 to about 25° C., preferably at about 2 to about 10° C., more preferably at about 4° C. The reaction can be carried out from about a few minutes to up to about 24 hours, preferably from about 12 to about 24 hours.

The material prepared according to the aforementioned alternative preparation (of the higher hydrated form) may be dried under vacuum and ambient temperatures or conventional drying at elevated temperature followed by re-exposure to ambient conditions (as detailed herein before) to furnish the lower hydrated form of sildenafil hemi-citrate as discussed hereinbefore.

Sildenafil Hemi-Citrate Dehydrated Form

As previously discussed herein another embodiment of the present invention provides a dehydrated form of sildenafil hemi-citrate. The present invention additionally provides a process for the preparation of said dehydrated form.

A preferred process herein for the preparation of the dehydrated form of sildenafil hemi-citrate, as defined herein, starting from sildenafil mono-citrate is:

(i) treatment of the sildenafil mono-citrate with a solution of an acidic buffer at a pH in the range of from about 4 to about 7.5;

(ii) isolation of solids at about the temperature of the stage (i) reaction, preferably from 0 to about 25° C.;

(iii) vacuum drying at ambient temperature or oven drying at about 80° C.; and

(iv) storage at 0% RH and ambient temperatures.

Highly preferred conditions for stages (i) and (ii) are as described previously for formation of the lower and higher hydrated forms.

Example 3 illustrates the process for the preparation of the dehydrated form of sildenafil hemi-citrate.

Further details of these methodologies are contained in the Experimental section hereinafter.

Formulations

The hydrated material(s) of the present invention may be used in freeze-drying, spray drying, or evaporative drying processes to provide a solid plug, powder, or film of crystalline or amorphous material. Microwave or radio frequency drying may also be used for this purpose.

The hydrated material(s) of the invention may be administered alone or in combination with other drugs and will generally be administered as a formulation in association with one or more pharmaceutically acceptable excipients. The term “excipient” is used herein to describe any ingredient other than the hydrated material(s) of the invention. The choice of excipient will to a large extent depend on the particular mode of administration.

Oral Administration

The hydrated material(s) of the invention may be administered orally. Oral administration may involve swallowing, so that the compound enters the gastrointestinal tract, or buccal or, sublingual administration may be employed by which the compound enters the blood stream directly from the mouth.

Formulations suitable for oral administration include solid formulations such as tablets, capsules containing particulates, liquids, or powders, lozenges (including

liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The hydrated material(s) of the invention may also be used in fast-dissolving, fast-disintegrating dosage forms such as those described in Expert Opinion in Therapeutic Patents, 11 (6), 981-986 by Liang and Chen (2001).

The composition of a typical tablet in accordance with the invention may comprise:

| Ingredient | % w/w |
|---|---|
| Sildenafil Hemi-Citrate Lower Hydrate | 10.00* |
| Microcrystalline cellulose | 64.12 |
| Lactose | 21.38 |
| Croscarmellose sodium | 3.00 |
| Magnesium stearate | 1.50 |

*Quantity adjusted in accordance with drug activity.

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and

sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25(2), 1-14 (2001). Other modified release formulations are described in U.S. Pat. No. 6,106,864.

Parenteral Administration

The hydrated material(s) of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of the hydrated material(s) of the present invention when used in the preparation of parenteral solutions may be increased by suitable processing, for example, the use of high-energy spray-dried dispersions (see WO 01/47495) and/or by the use of appropriate formulation techniques, such as the use of solubility-enhancing agents. The hydrated material(s) of the present invention may be present in either its crystalline or amorphous form in such formulations.

Formulations for parenteral administration may be formulated to be immediate and/or modified release. Modified release formulations include delayed-, sustained-, pulsed-, controlled dual-, targeted and programmed release.

Topical Administration

The hydrated material(s) of the invention may also be administered topically to the skin or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, J Pharm Sci, 88 (10), 955-958 by Finnin and Morgan (October 1999).

Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus hydrated material(s) of the invention may be formulated in a more solid form for administration as an implanted depot providing long-term release of the active compound.

Inhaled/Intranasal Administration

The hydrated material(s) of the invention can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorofluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

A suitable solution formulation for use in an atomiser using electrohydrodynamics to produce a fine mist may contain from 1 μg to 10 mg of the compound of the invention per actuation and the actuation volume may vary from 1 μl to 100 μl. A typical formulation may comprise a compound of formula (I), propylene glycol, sterile water, ethanol and sodium chloride. Alternative solvents, which may be used instead of propylene glycol, include glycerol and polyethylene glycol.

Capsules, blisters and cartridges (made, for example, from gelatin or HPMC) for use in an inhaler or insufflator may be formulated to contain a powder mix of the compound of the invention, a suitable powder base such as lactose or starch and a performance modifier such as l-leucine, mannitol, or magnesium stearate.

In the case of dry powder inhalers and aerosols, the dosage unit is determined by means of a valve that delivers a metered amount. Units in accordance with the invention are typically arranged to administer a metered dose or “puff” containing from about 0.05 to about 10 mg the compound of formula (I). The overall daily dose will typically be in the range 1 to 50 mg which may be administered in a single dose or, more usually, as divided doses throughout the day.

Formulations for inhaled/intranasal administration may be formulated to be immediate and/or modified release. Modified release formulations include delayed-, sustained-, pulsed-, controlled dual-, targeted and programmed release.

Rectal/Intravaginal Administration

The hydrated material(s) of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled dual-, targeted and programmed release.

Ocular/Andial Administration

The hydrated material(s) of the invention may also be administered directly to the eye or ear, typically in the form of drops of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

Enabling Technologies

The hydrated material(s) of the invention may be combined with soluble macromolecular entities such as cyclodextrin or polyethylene glycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Dosage

For oral and parenteral administration to human patients, the daily dosage level of the hydrated material(s) thereof will usually be from 10 to 500 mg (in single or divided doses).

Thus, for example, tablets or capsules of the hydrated material(s) may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, in the treatment of certain conditions (including MED and FSD), compounds may be taken as a single dose on an “as required” basis (i.e. as needed or desired).

These dosages are based on an average human subject having a weight of about 65 kg to 70 kg. The physician will readily be able to determine doses for subjects whose weight falls outside this range, such as infants and the elderly.

Generally, in humans, oral administration is the preferred route, being the most convenient and, for example in MED, avoiding the well-known disadvantages associated with intracavernosal (i.c.) administration. A preferred oral dosing regimen in MED for a typical man is from 25 to 250 mg of compound when required. In circumstances where the recipient suffers from a swallowing disorder or from impairment of drug absorption after oral administration, the drug may be administered parenterally, sublingually or buccally.

Thus the invention provides a pharmaeutical composition comprising a solid hydrated form of sildenafil hemi-citrate according to the present invention together with a pharmaceutically acceptable diluent or carrier.

It further provides a veterinary formulation comprising a novel hydrated solid form of sildenafil hemi-citrate according to the present invention together with a veterinarily acceptable diluent or carrier.

Medical Use

The hydrated material(s) of the invention are useful because they possesses pharmacological activity in animals, especially mammals, including humans.

According to a further aspect of the invention there is provided the compounds of the invention for use as pharmaceuticals, and for use as animal medicaments.

Thus the invention provides a pharmaceutical composition comprising a novel hydrated form of sildenafil hemi-citrate according to the present invention together with a pharmaceutically acceptable diluent or carrier.

The present invention further provides a veterinary formulation comprising a novel hydrated solid form of sildenafil hemi-citrate according to the present invention together with a veterinarily acceptable diluent or carrier.

According to a yet further aspect of the invention there is provided the use of the compounds of the invention in the manufacture of a medicament for the treatment of a medical condition in which a cGMP PDE (e.g. cGMP PDE5) is indicated. There is further provided the use of the compounds of the invention in the manufacture of a medicament for the treatment of a medical condition in which inhibition of a cGMP PDE (e.g. cGMP PDE5) is desirable.

By the term “treatment”, we include therapeutic (curative), palliative or prophylactic treatment.

The hydrated material(s) of the invention are thus expected to be useful for the curative, palliative or prophylactic treatment of mammalian sexual disorders.

In particular, the compounds are of value in the treatment of mammalian sexual dysfunctions such as male erectile dysfunction (MED), impotence, female sexual dysfunction (FSD), clitoral dysfunction, female hypoactive sexual desire disorder, female sexual arousal disorder, female sexual pain disorder or female sexual orgasmic dysfunction (FSOD) as well as sexual dysfunction due to spinal cord injury or selective serotonin re-uptake inhibitor (SSRI) induced sexual dysfunction but, clearly, will be useful also for treating other medical conditions for which a potent and selective cGMP PDE5 inhibitor is indicated. Such conditions include premature labour, dysmenorrhoea, benign prostatic hyperplasia (BPH), bladder outlet obstruction, incontinence, stable, unstable and variant (Prinzmetal) angina, hypertension, pulmonary hypertension, chronic obstructive pulmonary disease, coronary artery disease, congestive heart failure, atherosclerosis, conditions of reduced blood vessel patency, e.g. post-percutaneous transluminal coronary angioplasty (post-PTCA), peripheral vascular disease, stroke, nitrate induced tolerance, bronchitis, allergic asthma, chronic asthma, allergic rhinitis, diseases and conditions of the eye such as glaucoma, optic neuropathy, macular degeneration, elevated intra-occular pressure, retinal or arterial occulsion and diseases characterised by disorders of gut motility, e.g. irritable bowel syndrome (IBS).

Further medical conditions for which a potent and selective cGMP PDE5 inhibitor is indicated, and for which treatment with the compound of the present invention may be useful, include pre-eclampsia, Kawasaki's syndrome, nitrate tolerance, multiple sclerosis, diabetic nephropathy, neuropathy including autonomic and peripheral neuropathy and in particular diabetic neuropathy and symptoms thereof (e.g. gastroparesis), peripheral diabetic neuropathy, Alzheimer's disease, acute respiratory failure, psoriasis, skin necrosis, cancer, metastasis, baldness, nutcracker oesophagus, anal fissure, haemorrhoids, hypoxic vasoconstriction, diabetes, type 2 diabetes mellitus, the insulin resistance syndrome, insulin resistance, impaired glucose tolerance, as well as the stabilisation of blood pressure during haemodialysis.

Particularly preferred conditions include MED and FSD.

Thus, the invention provides a method of treating or preventing a medical condition for which a cGMP PDE5 inhibitor is indicated, in an animal (e.g. a mammal, including a human being), which comprises administering a therapeutically effective amount of the hydrated material(s) of the invention to a mammal in need of such treatment.

### EXAMPLES

The novel solid forms of sildenafil hemi-citrate as defined herein may be prepared and characterised according to the Examples and Experimental Methodology sections hereinafter.

PXRD Pattern

Whilst not wishing to be bound by any particular theory it is believed that a key identifier in the PXRD pattern, when generated using the methodology hereinafter, for the lower hydrated form of sildenafil hemi-citrate according to the present invention is the new peak at 5.9°. Similarly, it is believed that a key identifier in the PXRD pattern, when generated using the methodology hereinafter, for the higher hydrated form of sildenafil hemi-citrate according to the present invention is the new peak at 5.5°. Neither of these peaks are present in the PXRD pattern for the known “anhydrous” form of sildenafil mono-citrate (1:1).

Samples of the lower hydrated form of sildenafil hemi-citrate and the higher hydrated form of sildenafil hemi-citrate have been analysed by PXRD using a Siemens D5000 X-ray diffractometer using CuKα₁ radiation (wavelength=1.5406 Å) operating the tube at a voltage of 40 KV and a current of 40 mA. Samples were prepared by spreading a thin layer of the test compound as a dry powder on a low background silicon wafer. The wafer was rotated to reduce the effects of particle size and orientation and the samples analysed over an angular range of 2-40° two-theta using a scintillation counter. Samples were analysed at ambient temperatures (15-30° C.) and humidities (40-60% RH).

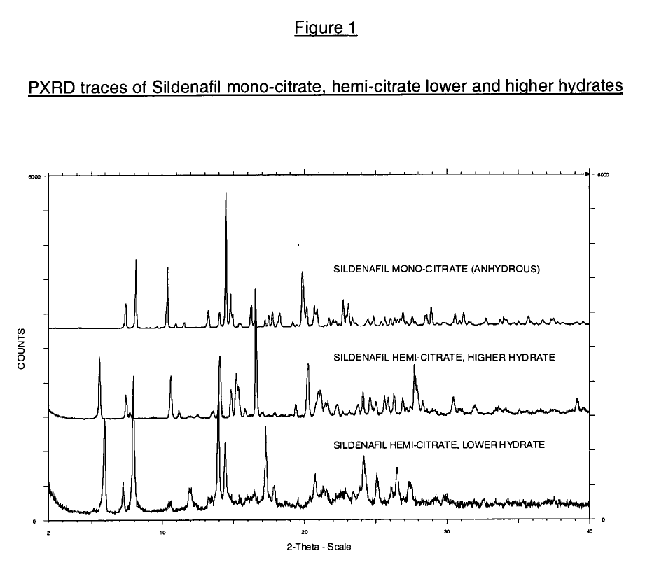

The PXRD patterns of isolated lower hydrate and isolated higher hydrate were compared to the PXRD pattern of anhydrous sildenafil mono-citrate. FIG. 1 illustrates the PXRD patterns for (a) anhydrous sildenafil mono-citrate; (b) sildenafil hemi-citrate lower hydrate; (c) sildenafil hemi-citrate higher hydrate.

FIG. 1 illustrates that the PXRD patterns for both hydrates are different both to each other and to the PXRD pattern for the mono-citrate. In particular both show different key (identifying) peaks versus the anhydrous form of sildenafil mono citrate. The key peaks at approximately 5.9° and 5.5° two-theta for the lower hydrate and higher hydrate respectively in FIG. 1 confirm the presence of the two separate new solid forms.

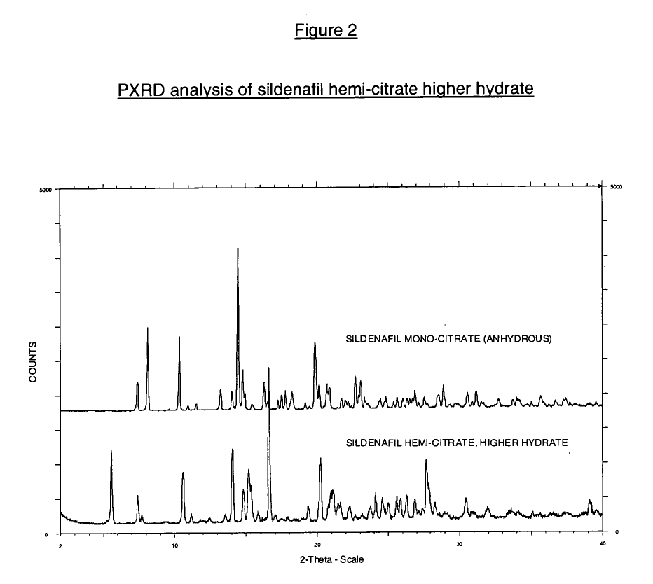

The solid formed in the citrate buffered solution of anhydrous sildenafil mono-citrate (the higher hydrate) was isolated and its PXRD pattern is illustrated in FIG. 2.

Table 1 illustrates the principal peaks in the PXRD pattern generated at ambient temperatures and RH for the higher hydrated form of sildenafil hemi-citrate according to the present invention. Principal peaks as defined herein are peaks having a relative intensity of greater than or equal to 10% of the most intense (highest) peak.

TABLE 1

| Angle (2-Theta) | Relative Intensity |
|---|---|
| 5.525 | 48.1 |
| 7.387 | 22.3 |
| 7.671 | 10 |
| 10.561 | 35.9 |
| 11.125 | 11.1 |
| 13.524 | 10.8 |
| 14.004 | 50.8 |
| 14.764 | 25.6 |
| 15.162 | 38.4 |
| 15.298 | 30.4 |
| 15.787 | 11.5 |
| 16.56 | 100 |
| 19.329 | 15.2 |
| 20.214 | 43.7 |
| 20.777 | 18.5 |
| 20.943 | 28.1 |
| 21.112 | 28.6 |
| 21.457 | 18.5 |
| 21.605 | 19.8 |
| 22.25 | 15.1 |
| 23.117 | 10.5 |
| 23.727 | 15.1 |
| 24.096 | 23.9 |
| 24.576 | 20.3 |
| 24.998 | 17 |
| 25.587 | 21.1 |
| 25.845 | 19.3 |
| 26.279 | 21.2 |
| 26.885 | 19 |
| 27.16 | 11.9 |
| 27.44 | 13.5 |
| 27.68 | 46.4 |
| 27.92 | 26.2 |
| 28.295 | 17.4 |
| 29.085 | 10.3 |
| 30.464 | 19.7 |
| 30.875 | 11.1 |
| 31.046 | 11.2 |
| 31.959 | 13.8 |
| 33.4 | 11.1 |
| 33.6 | 13.1 |
| 34.087 | 10.8 |
| 35.094 | 11.7 |
| 36.4 | 10.2 |
| 36.579 | 11.3 |
| 37.339 | 10.6 |
| 38.624 | 10.2 |
| 39.148 | 18.4 |
| 39.608 | 12.2 |

Thus the present invention provides a higher hydrated form of sildenafil hemi-citrate having a PXRD pattern substantially as defined in Table 1 wherein said PXRD pattern is generated using CuKα₁ radiation (wavelength=1.5406 Å) when measured according to the method described hereinbefore.

Table 2 provides a further characterisation of the higher hydrated form of sildenafil hemi-citrate wherein only the ten peaks (from Table 1) having the highest intensity are illustrated. Again the PXRD pattern illustrated in Table 2 was generated at ambient temperature and relative humidity (as defined hereinbefore for Table 1).

TABLE 2

| Angle (2-Theta) | Relative Intensity |
|---|---|
| 5.525 | 48.1 |
| 10.561 | 35.9 |
| 14.004 | 50.8 |
| 15.162 | 38.4 |
| 15.298 | 30.4 |
| 16.56 | 100 |
| 20.214 | 43.7 |
| 20.943 | 28.1 |
| 21.112 | 28.6 |
| 27.68 | 46.4 |

Thus the present invention provides a higher hydrated form of sildenafil hemi-citrate having a PXRD pattern substantially as defined in Table 2 wherein said PXRD pattern is generated using CuKα₁ radiation (wavelength=1.5406 Å) when measured according to the method described hereinbefore.

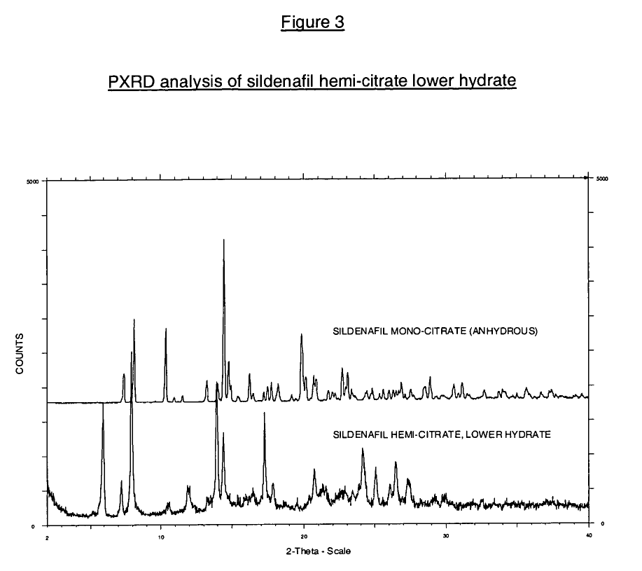

The solid formed by drying the higher hydrated form of sildenafil hemi-citrate followed by ambient storage thereof according to the process defined hereinbefore (i.e. the lower hydrated form) was isolated and its PXRD pattern is illustrated in FIG. 3.

Table 3 illustrates the principal peaks in the PXRD pattern generated at ambient temperatures and RH for the lower hydrated form of sildenafil hemi-citrate according to the present invention. Principal peaks as defined herein are peaks having a relative intensity of greater than or equal to 10% of the most intense (highest) peak.

TABLE 3

| Angle (2-Theta) | Relative Intensity |
|---|---|
| 5.899 | 71.1 |
| 7.19 | 25.9 |
| 7.905 | 100 |
| 10.523 | 14.4 |
| 11.905 | 21.2 |
| 13.25 | 16.9 |
| 13.893 | 83.7 |
| 14.371 | 50.9 |
| 15.104 | 15.1 |
| 15.373 | 17.9 |
| 15.948 | 18.1 |
| 16.431 | 20.1 |
| 17.221 | 64 |
| 17.812 | 25 |
| 18.668 | 14.2 |
| 19.492 | 15.8 |
| 20.38 | 18 |
| 20.708 | 32.6 |
| 21.318 | 23.5 |
| 21.5 | 22.4 |
| 22.256 | 17.8 |
| 22.493 | 22 |
| 22.86 | 23.2 |
| 23.415 | 21.3 |
| 23.86 | 24.7 |
| 24.145 | 45.1 |
| 25.075 | 33.6 |
| 25.565 | 16 |
| 26.086 | 23.9 |
| 26.506 | 36.8 |
| 27.356 | 27.2 |
| 28.24 | 15.9 |
| 29.02 | 15.3 |
| 29.207 | 17 |
| 29.799 | 16.5 |
| 29.926 | 19 |
| 30.361 | 13 |
| 31.01 | 13.2 |
| 31.891 | 14.6 |
| 32.556 | 15.3 |
| 33.073 | 12.5 |
| 33.4 | 16.2 |
| 33.768 | 12.8 |
| 34.219 | 13.7 |
| 34.38 | 13.3 |
| 34.752 | 13.9 |
| 35.332 | 14.1 |
| 36.115 | 13.3 |
| 37.122 | 15.1 |
| 37.514 | 15.7 |
| 38.69 | 13.3 |
| 39.009 | 12.8 |

Thus the present invention provides a lower hydrated form of sildenafil hemi-citrate having a PXRD pattern substantially as defined in Table 3 wherein said PXRD pattern is generated using CuKα₁ radiation (wavelength=1.5406 Å) when measured according to the method described hereinbefore.

Table 4 provides a further characterisation of the lower hydrated form of sildenafil hemi-citrate wherein only the ten peaks (from Table 3) which have the highest intensity are illustrated. Again the PXRD pattern illustrated in Table 4 was generated at ambient temperature and relative humidity (as defined hereinbefore for Table 1).

TABLE 4

| Angle (2-Theta) | Relative Intensity |
|---|---|
| 5.899 | 71.1 |
| 7.905 | 100 |
| 13.893 | 83.7 |
| 14.371 | 50.9 |
| 17.221 | 64 |
| 20.708 | 32.6 |
| 24.145 | 45.1 |
| 25.075 | 33.6 |
| 26.506 | 36.8 |
| 27.356 | 27.2 |

Thus the present invention provides a lower hydrated form of sildenafil hemi-citrate having a PXRD pattern substantially as defined in Table 4 wherein said PXRD pattern is generated using CuKα₁ radiation (wavelength=1.5406 Å) when measured according to the method described hereinbefore.

As will be appreciated by the skilled crystallographer, whilst the relative intensities of the various peaks within any of the PXRD Tables herein may vary due to a number of factors such as for example orientation effects of crystals in the X-ray beam or the purity of the material being analysed or the degree of crystallinity of the sample, the peak positions will remain substantially as defined in the Tables herein. The peak positions shown in Tables 1 to 4 (or in the Tables discussed hereinafter) may also shift in position depending upon the height of the sample in the X-ray beam as will be appreciated by the skilled crystallographer.

The skilled crystallographer will also appreciate that measurements using a different wavelength will result in different shifts according to the Bragg equation i.e. nλ=2d sin θ.

Such further PXRD patterns of the novel solid forms of sildenafil hemi-citrate according to the present invention, as well as mixtures thereof and mixtures of said novel forms with either mono-citrate or sildenafil free-base, generated by use of alternative wavelengths are considered to be alternative representations of the PXRD patterns of the crystalline materials of the present invention, or further representations comprising crystalline materials according to the present invention and as such are considered to be within the scope of the present invention.

NMR Analysis

The novel hydrated forms of sildenafil hemi-citrate according to the present invention were analysed by ¹H NMR. As the NMR studies were carried out in solution the patterns obtained for both hydrates were essentially the same.

From the NMR results the stoichiometry of the sildenafil and the citrate portions in both hydrates has been determined to be 1:0.5.

Thus the present invention additionally provides a novel lower hydrated solid form of sildenafil hemi-citrate wherein the relative amounts of sildenafil (drug) to citric acid (counter ion) are present in a molar ratio of 1:0.5.

Thus the present invention additionally provides a novel higher hydrated solid form of sildenafil hemi-citrate wherein the relative amounts of sildenafil (drug) to citric acid (counter ion) are present in a molar ratio of 1:0.5.

The assignment of the ¹H NMR spectrum of the hydrated forms of sildenafil hemi-citrate (the lower hydrate and the higher hydrate) according to the present invention was based on the known assignment of sildenafil mono-citrate. The analysis was carried out using a Varian INOVA NMR spectrometer, operating at 500 MHz for proton (¹H) detection. The solvent used was DMSO-d6 and the operating temperature was 30° C. The acquisition parameters were set up as such to maximise the accuracy of the integration levels. The ratio of sildenafil:citrate, in the solid forms according to the present invention, was found to be approximately 1:0.5, by comparison of the integration values for the 2′ and 4′ protons in the citrate anion with the integration values for the protons in the sildenafil cation. The ¹H NMR data for the lower hydrated form (and by correspondence thereto, the higher hydrated form) are summarised in the listings detailed in Table 5.

TABLE 5
¹H NMR of the Lower Hydrate of Sildenafil
Hemi-Citrate in DMSO-d6

| Atom | δ_{H} (ppm) | No. of H's | Mult. | J (Hz) |
|---|---|---|---|---|
| 13 | 0.94 | 3 | T | 7 |
| 21 | 1.34 | 3 | T | 7 |
| 12 | 1.75 | 2 | M | — |
| 29 | 2.21 | 3 | S | — |
| 25, 27 | 2.45 | 4 | M | — |
| 2′, 4′ | 2.62 | 1 | D | 15 |
| 2′, 4′ | 2.71 | 1 | D | 15 |
| 11 | 2.78 | 2 | T | 7 |
| 24, 28 | 2.96 | 4 | M | — |
| 10 | 4.15 | 3 | S | — |
| 20 | 4.23 | 2 | Q | 7 |
| 18 | 7.36 | 1 | D | 9 |
| 17 | 7.82 | 1 | dd | 2, 9 |
| 15 | 7.88 | 1 | D | 2 |
| 5 | 12.06 | 1 | S | — |

Peak listing ¹H NMR (500 MHz, DMSO-D6) δ ppm 0.94 (t, J=7 Hz, 3H) 1.34 (t, J=7 Hz, 3H) 1.75 (m, 2H) 2.21 (s, 3H) 2.45 (m, 4H) 2.62 (d, J=15 Hz, 1H) 2.71 (d, J=15 Hz, 1H) 2.78 (t, J=7 Hz, 2H) 2.96 (m, 4H) 4.15 (s, 3H) 4.23 (q, J=7 Hz, 2H) 7.36 (d, J=9 Hz, 1H) 7.82 (dd, J=2 & 9 Hz, 1H) 7.88 (d, J=2 Hz, 1H) 12.06 (s, 1H).

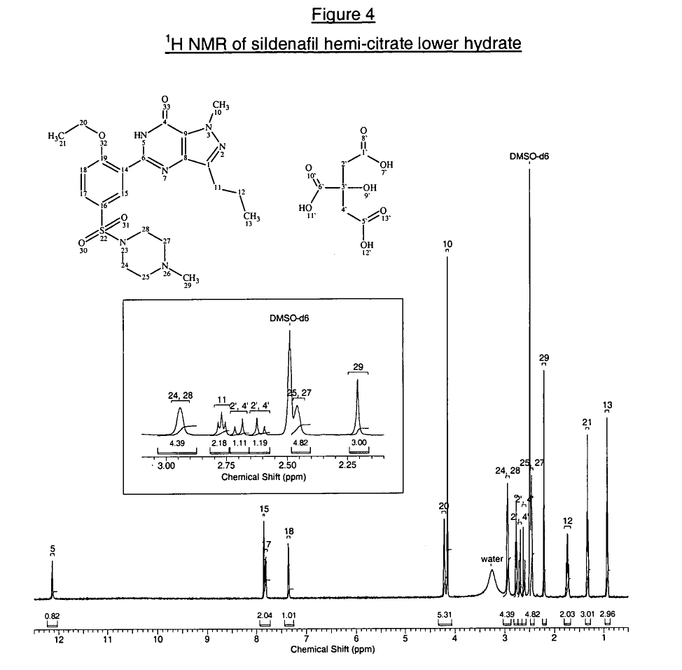

FIG. 4 illustrates the ¹H NMR pattern for the lower hydrated form of sildenafil hemi-citrate.

Dynamic Vapour Sorption (DVS)

The moisture sorption of the hydrated forms of sildenafil hemi-citrate was measured using a Surface Measurement Systems Ltd. Dynamic Vapour Sorption (DVS) Automated Water Sorption Analyser. A sample of the higher hydrate was initially equilibrated at about 45% RH and then analysed from 0 to 90% RH in relative humidity steps of 10% (exposure time of 6 hours per step). A second analysis was carried out from 35 to 65% RH in 5% RH steps to gain more detail in this humidity range (exposure time variable, weight stability of 0.0003%/minute gained for each steps). The temperature was maintained at 30° C. throughout the equilibration and analysis stages.

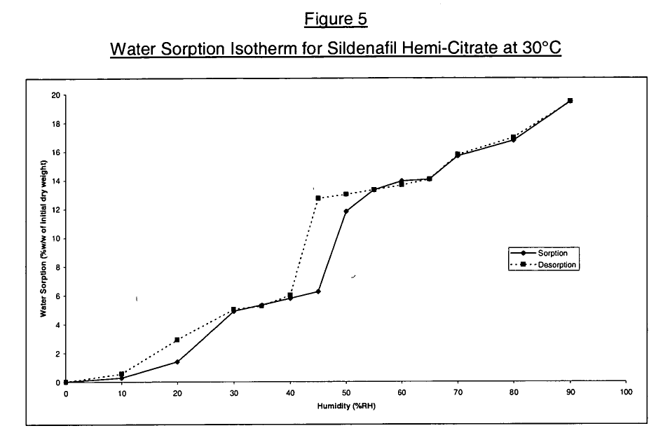

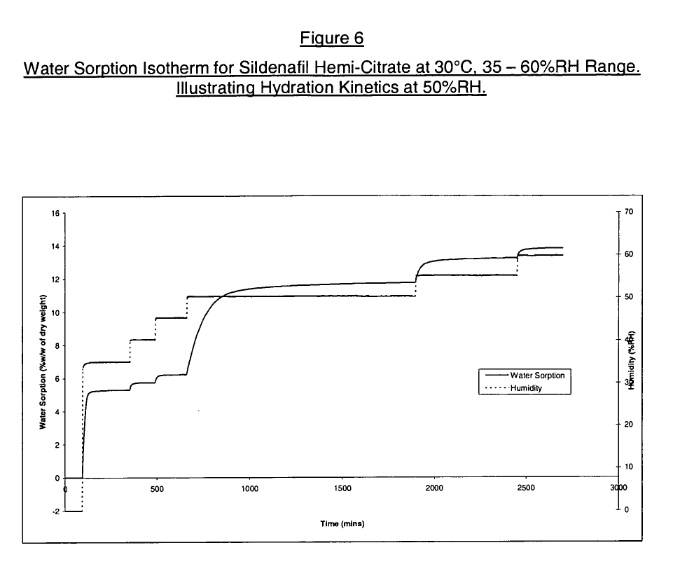

Sildenafil hemi-citrate is a hygroscopic material. The isotherm is represented in FIG. 5. At humidities greater than 55% RH the higher hydrate is present and from 30 to 40% RH the lower hydrate is present and at 0% RH the dehydrated form is present. Hysteresis occurs between the sorption and desorption isotherms in the 10 to 30 and 40 to 55% RH regions. This is classically seen for hydrates where kinetics associated with hydration and dehydration result in the events occurring at different humidities. There are slow kinetics associated with the rate of sorption (see FIG. 6) at 50% RH (illustrated by an equilibration time of 21 hours). Further analysis has indicated that kinetics of desorption are also slow at 40% RH (an equilibration time of 24 hours has been observed).

Encompassed within the spirit and scope of the present invention are any polymorphic variants and changes due to variation of the temperature and humidity conditions used during the PXRD analysis of the hydrated and “dehydrated” hydrate forms of sildenafil hemi-citrate herein.

TGA Analysis

TGA analysis has been performed on the higher hydrated form of sildenafil hemi-citrate as described herein.

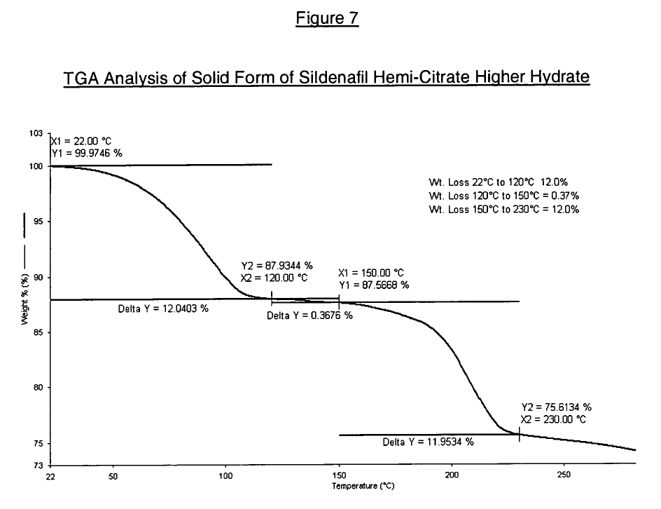

A TGA trace showing the relative % weight loss versus temperature for the solid form of the present invention is illustrated in FIG. 7. This trace was obtained using a Perkin-Elmer Pyris 1 TGA instrument. The sample, ideally about 5 to about 10 mg, was heated at 20° C./minute from ambient to about 300° C. with a purge gas of nitrogen.

FIG. 7 illustrates that a weight loss of about 12% was observed when the sample was heated from ambient to about 120° C. Such weight loss, calculated as moles of water per mole of anhydrous sildenafil hemi-citrate (1:0.5) indicates that the new solid form has approximately 4.3 moles of water per mole of hemi-citrate and as such may be broadly referred to as a tetra (4) hydrated form of sildenafil hemi-citrate.

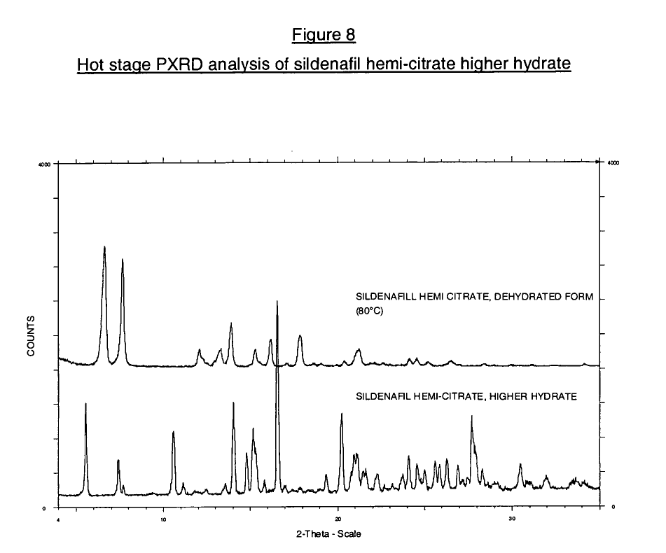

The solid form remaining after the first weight loss is regarded as the “dehydrated hydrate”/anhydrous form identified via hot stage PXRD and as illustrated in FIG. 8. Further heating of this “dehydrated hydrate” form results in a second weight loss of about 12%, observed at temperatures in excess of 150° C. This has been attributed to degradation of the citrate ion.

Thus, according to a further aspect the present invention provides a novel “dehydrated” anhydrous form of sildenafil hemi-citrate and a process for its preparation. Such novel anhydrous form has been described hereinbefore as a “dehydrated” form of the hydrated forms of sildenafil hemi-citrate. Dehydrated hydrates are discussed in Stephenson, G. A., Groleau, E. G., Kleiman, R. L., Xu, W., Rigsbee, D. R., J. Pharm. Sci. 87(5) (1998), pages 536-542 incorporated herein by reference.

The presence of said dehydrated hydrate has been discussed in relation to TGA analysis. The structure of the dehydrated hydrate has been identified using simultaneous scanning for structure via hot stage PXRD of the higher hydrated form of sildenafil hemi-citrate and PXRD under vacuum of the higher hydrated form of sildenafil hemi-citrate.

Hot stage powder X-ray diffraction was been performed using a Bruker D8 X-ray powder diffractometer fitted with Goebel mirrors and a Braun position sensitive detector. A sample of the higher hydrated form of sildenafil hemi-citrate was presented on a silicon wafer and heated to 80° C. using an Ansyco heating and humidity stage. The radiation used was CuKα₁ radiation (wavelength=1.5406 Å) and analysis was performed over an angular range of 4-35° two-theta. Table 6 below gives the peak positions for the PXRD pattern of the dehydrated form produced at 80° C. and illustrated in FIG. 8.

TABLE 6
peak positions of dehydrated form at 80° C.

| Angle (two theta) | Intensity |
|---|---|
| 6.594 | 100 |
| 7.628 | 90.4 |
| 12.041 | 19.5 |
| 13.243 | 18.1 |
| 13.865 | 40.2 |
| 15.271 | 18.9 |
| 16.151 | 26.6 |
| 17.099 | 8.1 |
| 17.859 | 29.7 |
| 18.624 | 8.3 |
| 19.061 | 7.9 |
| 20.384 | 9.8 |
| 21.199 | 19.2 |
| 24.123 | 11.2 |
| 24.544 | 10.8 |
| 25.192 | 8.6 |

Table 7 provides the top ten peaks of the dehydrated form at 80° C.

TABLE 7

| Angle (two theta) | Intensity |
|---|---|
| 6.594 | 100 |
| 7.628 | 90.4 |
| 12.041 | 19.5 |
| 13.243 | 18.1 |
| 13.865 | 40.2 |
| 15.271 | 18.9 |
| 16.151 | 26.6 |
| 17.859 | 29.7 |
| 21.199 | 19.2 |
| 24.123 | 11.2 |

Thus according to further aspects the present invention additionally provides a dehydrated form of sildenafil hemi-citrate having a PXRD pattern essentially as illustrated in either Table 6 or Table 7 when measured according to the methods detailed herein before.

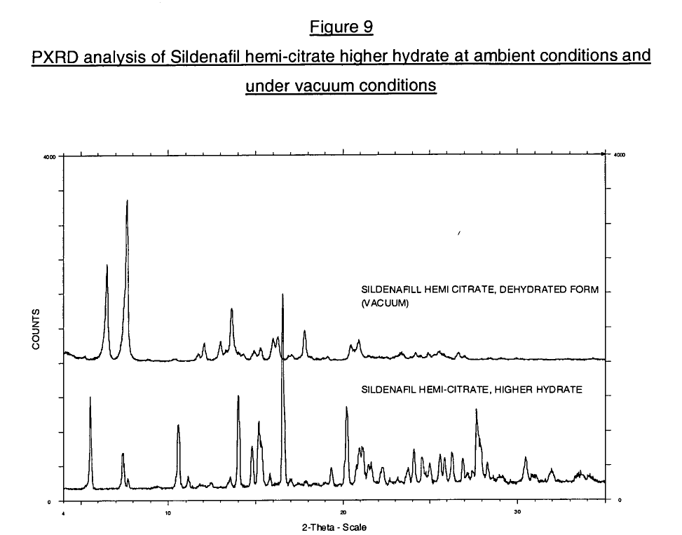

The characteristics of the dehydrated form were also assessed using PXRD analysis under vacuum (<10 mBar) by dehydrating a sample of the higher hydrate under vacuum and recording it's PXRD pattern using a Bruker D8 X-ray powder diffractometer fitted with Goebel mirrors and a Braun position sensitive detector. The radiation used was CuKα₁ radiation (wavelength=1.5406 Å) and analysis was performed over an angular range of 4-35° two-theta. The PXRD pattern observed via this analysis is illustrated in FIG. 9.

Table 8 provides the peak positions of the dehydrated hydrate.

TABLE 8
Peak positions for dehydrated hydrate

| Angle (2-Theta) | Relative Intensity |
|---|---|
| 5.188 | 6.6 |
| 6.473 | 61.6 |
| 7.624 | 100 |
| 10.382 | 5.4 |
| 11.702 | 8.7 |
| 12.046 | 14.9 |
| 12.965 | 15.4 |
| 13.621 | 35.3 |
| 14.263 | 7.9 |
| 14.902 | 10.1 |
| 15.28 | 12 |
| 15.986 | 17.5 |
| 16.239 | 18.2 |
| 17.032 | 7.7 |
| 17.795 | 21.9 |
| 18.139 | 6.5 |
| 19.126 | 6.7 |
| 20.452 | 13.7 |
| 20.897 | 16.1 |
| 21.483 | 7 |
| 23.352 | 8.9 |
| 24.182 | 8.6 |
| 24.926 | 8.7 |
| 25.526 | 9.1 |
| 26.635 | 8.6 |
| 26.98 | 6.7 |

It is believed that the peak at 5.188 is attributable to a residual amount of the free base.

Thus the present invention provides a further novel form of sildenafil hemi-citrate having a PXRD pattern substantially as defined in Table 8 wherein said PXRD pattern is generated using CuKα₁ radiation (wavelength=1.5406 Å) and under vacuum when measured according to the method described hereinbefore.

Table 9 shows the ten most intense peaks (from Table 8).

TABLE 9

| Angle (2-Theta) | Relative Intensity |
|---|---|
| 6.473 | 61.6 |
| 7.624 | 100 |
| 12.046 | 14.9 |
| 12.965 | 15.4 |
| 13.621 | 35.3 |
| 15.986 | 17.5 |
| 16.239 | 18.2 |
| 17.795 | 21.9 |
| 20.452 | 13.7 |
| 20.897 | 16.1 |

Thus the present invention provides a further novel form of sildenafil hemi-citrate having a PXRD pattern substantially as defined in Table 9 wherein said PXRD pattern is generated using CuKα₁ radiation (wavelength=1.5406 Å) and under vacuum when measured according to the method described hereinbefore.

Preparations

Preparation 1-5-[2-Ethoxy-5-(4-methylpiperazin-1-ylsulphonyl) phenyl]-1-methyl-3-n-propyl-1,6-dihydro-7H-pyrazolo[4,3-d]pyrimidin-7-one mono-citrate

A solution of 5-[2-ethoxy-5-(4-methyl piperazin-1-ylsulphonyl)phenyl]-1-methyl-3-n-propyl-1,6-dihydro-7H-pyrazolo[4,3-d]pyrimidin-7-one in a suitable organic solvent was heated up to the reflux temperature of the solvent whereupon an aqueous solution of citric acid was added. The product was isolated from the solution via filtration as a white crystalline solid, m.p. around 195° C. Found: C,50.51; H,5.60; N,12.59. C₂₂H₃₀N₆O₄S; C₆H₈O₇ requires C,50.44; H,5.75; N,12.61%.

Preparation 2—Formation of Sildenafil Citrate Lower Hydrate from Sildenafil Monocitrate (Anhydrous Sildenafil Citrate)

Anhydrous sildenafil mono-citrate (250 mg) in 2 ml of 0.2M phosphoric acid buffer (prepared according to the procedure hereinafter) was slurried (using a rotating bed) at about 4° C. for about 72 hours. The resultant solid was isolated using vacuum filtration and allowed to dry at ambient temperature and humidity. The sample was analysed by PXRD, ¹H. NMR and TGA techniques such as those described hereinbefore and via and EGA techniques as discussed hereinafter.

Preparation of pH 4.76 Acetic Acid Buffer [0.2 Molar]

12.01 grams of Glacial Acetic Acid was weighed into a 1 litre beaker and approximately 400 mls of water was carefully added. The pH was adjusted to 4.76 with concentrated NaOH [carbonate free]—‘CONVOL’ concentrate is suitable. The solution was transferred quantitatively to a 1 litre volumetric flask and made up to 1 litre. The buffer solution was divided, in 25 ml aliquots, to 50 ml vials and sealed prior to autoclaving the vials and storing at 4° C.

‘ANALAR’ grade, or similar, chemicals and milliq de-ioised water were used in the preparation of this buffer. When using the buffer it can, if necessary, be diluted to 1:20 to give an in-use concentration of 0.01M and NaCl [0.67% W/V] can be added to maintain chloride ion concentration and ionic strength.

Preparation of pH 6.4 Citric Acid Buffer [0.2 Molar]

This buffer can be prepared according to the general procedure detailed in Preparation 1. Weigh 38.42 grams of Anhydrous Citric Acid into a 1 litre beaker and add approximately 150 mls of water (maximum). 42.02 grams of Citric Acid Monohydrate can alternatively be used. Adjust the pH to 6.4 with concentrated NaOH [carbonate free]—‘CONVOL’ concentrate is suitable. The solution can be transferred into vials as detailed herein before.

Preparation of pH 7.20 Phosphoric Acid Buffer [0.2 Molar]

This buffer can be prepared according to the general procedure detailed in Preparation 1. Weigh 19.60 grams of concentrated orthophosphoric acid into a 1 litre beaker and carefully add approximately 200 mls of water (maximum). Adjust the pH to 7.20 with concentrated NaOH [carbonate free]—‘CONVOL’ concentrate is suitable. The solution can be transferred into vials as detailed herein before.

### Example 1

### Formation of Sildenafil Citrate Higher Hydrate from Sildenafil Monocitrate (Anhydrous Sildenafil Citrate)

To 2 g of sildenafil monocitrate (anhydrous) 32 mL of citric acid buffer (pH 6.4) at 4° C. was added. The mixture was agitated (via stirring) for a period of about 12 hours at 4° C. The resultant solid material was isolated via vacuum filtration (Buchner funnel) at the temperature of the reaction (4° C.), and then allowed to dry at approximately 55% RH, under ambient temperatures for about 24 hours. The final hydrate was assessed via PXRD and TGA as detailed hereinbefore.

### Example 2

### Formation of Sildenafil Citrate Lower Hydrate from Sildenafil Monocitrate (Anhydrous Sildenafil Citrate)

To 2 g of sildenafil monocitrate (anhydrous) 32 mL of citric acid buffer (pH 6.4) at 4° C. was added. The mixture was agitated (via stirring) for a period of about 12 hours at 4° C. The resultant solid material was isolated via vacuum filtration (Buchner funnel) at the temperature of the reaction (4° C.). The filtered solid was dried under vacuum (ca. 10 mbar) for a period of about 12 hours. The dried solid was then exposed to approximately 40% RH at ambient temperatures for a period of about 24 hours to furnish the desired hydrated material which was assessed using PXRD and ¹H NMR as detailed hereinbefore.

### Example 3

### Formation of Sildenafil Citrate Dehydrated Hydrate

To 2 g of sildenafil monocitrate (anhydrous) 32 mL of citric acid buffer (pH 6.4) at 4° C. was added. The mixture was agitated (via stirring) for a period of about 12 hours at 4° C. The resultant solid material was isolated via vacuum filtration (Buchner funnel) at the temperature of the reaction (4° C.). The dehydrated form was produced by drying the solid in a vacuum oven (ca. 10 mbar) at ambient temperature. To minimise conversion to the lower hydrated form the dehydrated form was stored at low humidity (and preferably under vacuum).

In Examples 1, 2 and 3 any quantity of sildenafil monocitrate (anhydrous) and citric acid buffer can be used, provided that a concentration of approximately 62.5 mg/mL is attained.

According to a further embodiment the present invention additionally provides solid forms of sildenafil citrate when prepared according to the alternative process of Example 4.

### Example 4

### Formation of Sildenafil Citrate Higher Hydrate from Sildenafil Monocitrate (Anhydrous Sildenafil Citrate)

Anhydrous sildenafil citrate (250 mg) in 2 ml of 0.2M phosphoric acid buffer (prepared according to the procedure hereinafter) was slurried (using a rotating bed) at about 4° C. for about 72 hours. The resultant solid was isolated using vacuum filtration and allowed to dry at ambient temperature and humidity. The sample was analysed by PXRD, ¹H. Nmr, TGA and EGA as detailed hereinafter and characterised as containing the higher hydrated form of sildenafil citrate according to the present invention. Preliminary analysis as detailed hereinafter indicated that the ratio of sildenafil:citrate in the solid material obtained from the process of Example 4 was about 1:0.75 this has since been confirmed to be a mixture which contains the higher hydrated form of sildenafil hemi-citrate (1:0.5) as defined herein.

Samples of the solid material obtained from the process of Example 4 have been analysed by PXRD using a Bruker D8 Advance X-ray diffractometer using CuKα₁ radiation (wavelength=1.5406 Å) operating the tube at a voltage of 40 KV and a current of 40 mA. Samples were prepared by spreading a thin layer of either the dry powder or a slurry of the material of interest on a low background silicon wafer. The samples were irradiated in the X-ray beam which was made parallel using Goebel mirrors with a slit size of 0.2 mm and analysed over an angular range of 4-35° two-theta using a Braun position sensitive detector fitted with radial soller slits. Samples were analysed at ambient temperatures (15-30° C.) and humidities (40-60% RH).

The PXRD patterns of mixtures of anhydrous sildenafil citrate in buffer(s) that had been slurried at about 4° C. for about 72 hours (according to the process detailed above) were compared to the PXRD pattern of anhydrous sildenafil.

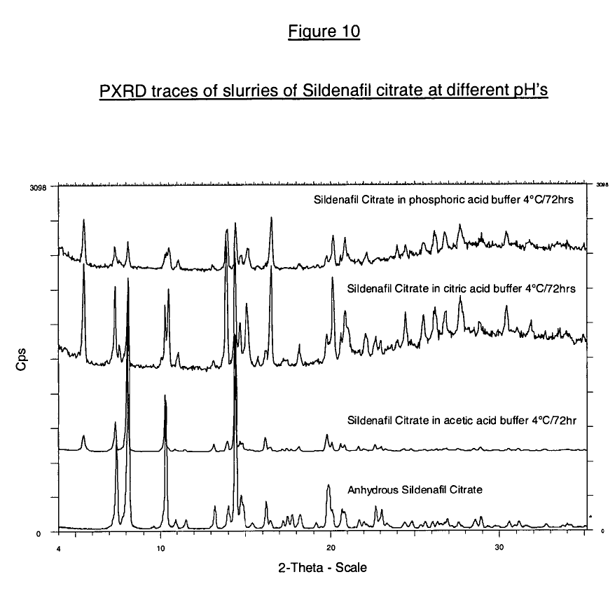

FIG. 10 illustrates the PXRD patterns for (a) anhydrous sildenafil citrate (non-buffered); (b) sildenafil citrate in phosphate buffer at about pH7.3; (c) sildenafil citrate in citrate buffer at about pH 6.6; and (d) sildenafil citrate in acetate buffer at about pH 4.72.

FIG. 10 illustrates that the buffered slurries of the citrate, acetate and phosphate show additional peaks versus the anhydrous form of sildenafil citrate. The peaks at approximately 5.5° and 16.5° two-theta for the buffered solution traces in FIG. 10 provide evidence of the presence of another solid form.

It is proposed herein that the difference in rate of conversion (to the hydrated form) across the pH range is likely due to the different chemical interactions due to the pKa of the sildenafil molecule.

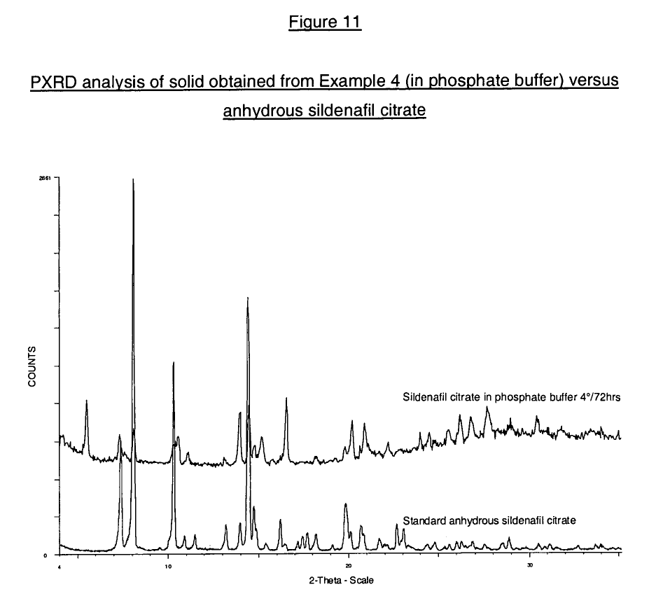

The solid formed in the phosphate buffered solution of anhydrous sildenafil citrate was isolated and its PXRD pattern is illustrated in FIG. 11.

Table 10 illustrates the principal peaks in the PXRD pattern generated at ambient temperatures and RH for the solid material obtained from the process of Example 4.

TABLE 10

| Angle | Intensity % |
|---|---|
| 2-Theta ° | % |
| 4.138 | 66.9 |
| 5.48 | 98.3 |
| 7.307 | 66.7 |
| 8.093 | 72.3 |
| 10.321 | 60.5 |
| 10.532 | 65 |
| 11.068 | 50.8 |
| 13.123 | 44.2 |
| 13.926 | 86.4 |
| 14.462 | 93.3 |
| 14.755 | 56.2 |
| 15.132 | 64.6 |
| 15.71 | 43.9 |
| 16.537 | 100 |
| 18.163 | 46.5 |
| 19.777 | 55.5 |
| 20.177 | 78.9 |
| 20.888 | 76.5 |
| 22.19 | 59 |
| 24.018 | 67.6 |
| 24.505 | 67.4 |
| 24.804 | 60.6 |
| 25.557 | 69.9 |
| 26.207 | 83.7 |
| 26.798 | 81.9 |
| 27.698 | 90.8 |
| 28.964 | 80.9 |
| 30.436 | 82.7 |
| 31.807 | 73.3 |
| 34.055 | 71.4 |
| 34.98 | 69 |
| 7.554 | 7.5 |
| 20.607 | 10.1 |

Thus the present invention provides material having a PXRD pattern substantially as defined in Table 10 wherein said PXRD pattern is generated using CuKα₁ radiation (wavelength=1.5406 Å) when measured according to the method described hereinbefore.

Table 12 provides a further characterisation of the material from the process of Example 4 wherein only those peaks having an intensity greater than 80% are illustrated. Again the PXRD pattern illustrated in Table 12 was generated at ambient temperature and relative humidity (as defined hereinbefore for Table 10).

TABLE 12

| Angle | Intensity % |
|---|---|
| 2-Theta ° | % |
| 5.48 | 98.3 |
| 13.926 | 86.4 |
| 14.462 | 93.3 |
| 16.537 | 100 |
| 26.207 | 83.7 |
| 26.798 | 81.9 |
| 27.698 | 90.8 |
| 28.964 | 80.9 |
| 30.436 | 82.7 |
| 26.207 | 83.7 |

Thus the present invention provides a material having a PXRD pattern substantially as defined in Table 12 wherein said PXRD pattern is generated using CuKα₁ radiation (wavelength=1.5406 Å) when measured according to the method described hereinbefore.

NMR Analysis

The solid material obtained from the process of Example 4 was analysed by ¹H NMR. From the NMR results the relative stoichiometry of the sildenafil and the citrate portions in this material has been determined to be 1:0.75.

The assignment of the ¹H NMR spectrum of the material from the process of Example 4 was based on the known assignment of sildenafil citrate. The analysis was carried out using a Varian INOVA NMR spectrometer, operating at 500 MHz for proton (1H) detection. The acquisition parameters were set up as such to maximise the accuracy of the integration levels. The ratio of sildenafil:citrate was found to be approximately 1:0.75, by comparison of the integration values for the 2′ and 4′ protons in the citrate anion with the integration values for the protons in the sildenafil cation. The ¹H NMR data is summarised in the listing detailed hereinafter and in Table 13.

TABLE 13
¹H NMR data for Solid from Example 4 in DMSO-d6

| | δH | | | |
|---|---|---|---|---|
| Atom | (ppm) | J (Hz) | Multiplicity | H's |
| 13 | 0.93 | 7 | triplet | 3 |
| 21 | 1.32 | 7 | triplet | 3 |
| 12 | 1.74 | 7 | sextet | 2 |
| 29 | 2.19 | — | singlet | 3 |
| 25, 27 | 2.43 | — | multiplet | 4 |
| 2′, 4′ | 2.59 | 15 | doublet | 1.5 |
| 2′, 4′ | 2.68 | 15 | doublet | 1.5 |
| 11 | 2.77 | 7 | triplet | 2 |
| 24, 28 | 2.93 | — | multiplet | 4 |
| 10 | 4.14 | — | singlet | 3 |
| 20 | 4.21 | 7 | quartet | 2 |
| 18 | 7.36 | 9 | doublet | 1 |
| 17 | 7.81 | 2, 9 | doublet of | 1 |
| | | | doublets | |
| 15 | 7.85 | 2 | doublet | 1 |
| 5 | 12.16 | — | singlet | 1 |

Peak listing: ¹H NMR (500 MHz, DMSO-d6) δ ppm 0.93 (t, J=7 Hz, 3H) 1.32 (t, J=7 Hz, 3H) 1.74 (m, J=7 Hz, 2H) 2.19 (s, 3H) 2.43 (m, 4H) 2.59 (d, J=15 Hz, 1.5H) 2.68 (d, J=15 Hz, 1.5H) 2.77 (t, J=7 Hz, 2H) 2.93 (m, 4H) 4.14 (s, 3H) 4.21 (q, J=7 Hz, 2H) 7.36 (d, J=9 Hz, 1H) 7.81 (dd, J=9, 2 Hz, 1H) 7.85 (d, J=2 Hz, 1H) 12.16(s, 1H)

DSC Analysis

DSC analysis indicated that the solid obtained from the process of Example 4 loses water on heating and that the citrate ion degraded on melting of the resultant “dehydrated” hydrate at 175° C. These observations have been confirmed by EGA analysis as discussed below.

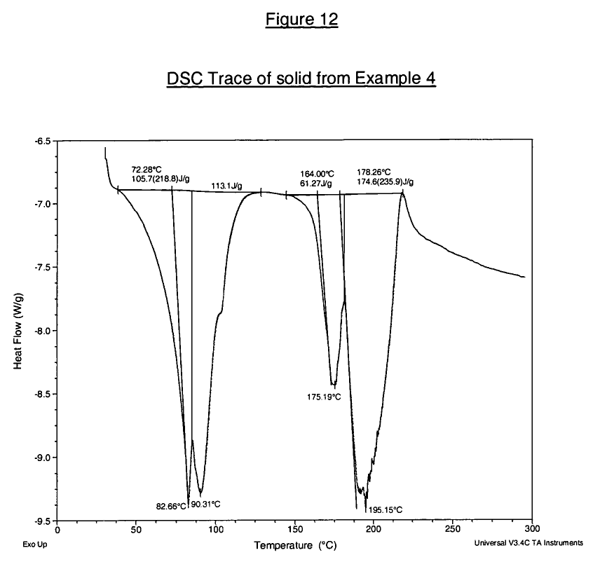

Thermal analysis of a sample of the solid obtained from the process of Example 4 by DSC was performed using a TA Instruments Q1000 DSC heating at 20° C./min with a purge gas of nitrogen and crimped aluminium pans. The results of the DSC analysis are illustrated in FIG. 12.

FIG. 12 illustrates the heatflow relative to temperature and shows principal events at about 90° C. and at about 175° C. The first two events are attributable to a dehydration event and the second event, at about 175° C., is attributable to a melt of the resultant “anhydrous form of the hydrate” with degradation of the citrate ion following the melt (at 195.1° C.).

This “anhydrous form” of the solid form of the present invention can be regarded as a “dehydrated” form of the solid form of the present invention and is not to be considered equivalent to anhydrous sildenafil citrate. The melting point of the anhydrous form the “dehydrated hydrate” is 175° C. and that of anhydrous sildenafil citrate is 202° C. Further investigations into this “dehydrated hydrate”/anhydrous form are discussed hereinafter.

TGA Analysis

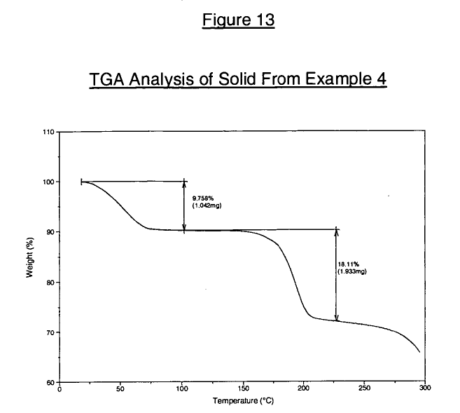

A TGA trace showing the relative % weight loss versus temperature for the solid obtained from the process of Example 4 is illustrated in FIG. 13. This trace was obtained using a TA Instruments High Resolution 2910 instrument. The sample, ideally about 5 to about 10 mg, was heated at 20° C./minute from ambient to 200° C. with a purge gas of nitrogen.

FIG. 13 illustrates that a weight loss of about 10% was observed when the sample was heated from ambient to about 80° C. Such weight loss, calculated as a potential water loss from anhydrous sildenafil citrate (1:0.75) salt indicates that the new solid form is present as a tetra (4) hydrate salt.

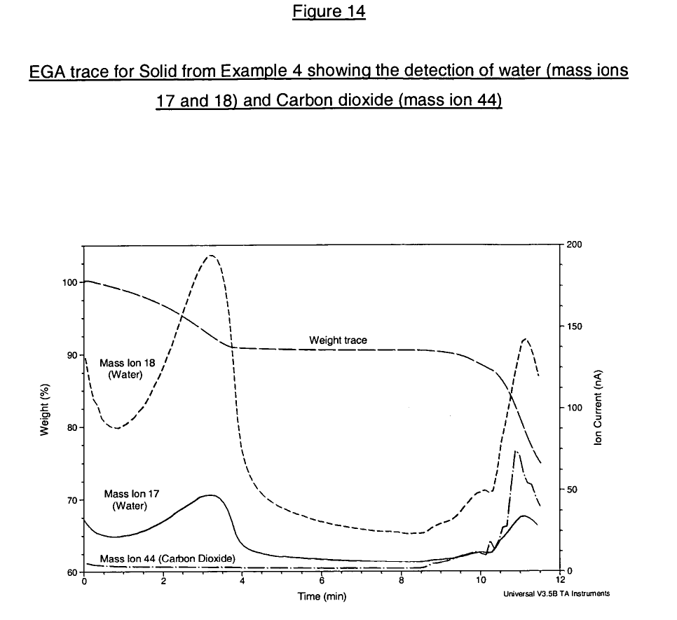

The solid form remaining after the first weight loss is regarded as the “dehydrated hydrate”/anhydrous form identified via DSC and illustrated in FIG. 14. Further heating of this “dehydrated hydrate” form results in a second weight loss of about 18%, observed at temperatures in excess of 150° C.

EGA Analysis

To further investigate the processes occurring during the heating ramp Evolved Gas Analysis (EGA) was performed on the solid obtained from the process of Example 4. This technique monitors the weight lost during heating whilst simultaneously identifying the components of the evolved gas using mass spectrometry (MS). This provides data relating to any desolvation/degradation processes.

To determine the composition of the matter lost on heating, the exhaust gas from TGA apparatus was fed into a quadropole mass spectrometer. This TGA trace (illustrated in FIG. 14) was obtained using a TA Instruments Q50 TGA with platinum pans. The sample, ideally from about 5 mg to about 10 mg, was heated at 20° C./minute from ambient to 200° C. with a purge gas of helium. The exhaust gas was analysed by a Pfeifer Thermostar Mass spectrometer working in “trend” mode. Ions 17 and 18 corresponding to water and 44 for carbon dioxide were monitored. Ion currents, which are traces displaying the weight loss versus temperature, for each of the specific ions are also illustrated in FIG. 14.

FIG. 14 shows a trace of the TGA (see FIG. 13) combined with the signals for mass ions 17 & 18 (water) and 44 (carbon dioxide).

In line with the initial TGA analysis (illustrated in FIG. 13 as discussed hereinbefore) FIG. 14 also illustrates that a % weight loss of about 10% is observed during the initial heating stage (at from about RT to about 80° C.).

The traces related to the relative increase in the release of mass ions 17 and 18 indicate that release of water have been detected (by MS) during this lower temperature initial loss period. Based on these results this first weight loss can be attributed to release of the bound water from within the crystal lattice.

Analysis of the relative release of mass ions 17, 18 and 44 during the second period of weight loss, at higher temperatures (corresponding to about 18%) at from about 150° C. to about 200° C. indicates that both water and carbon dioxide are emitted. This is consistent with weight loss attributable to the degradation of the citric acid in the sample of the “dehydrated” form of the sildenafil citrate tetrahydrate.

The presence of a dehydrated hydrate obtained from the solid obtained from the process of Example 4 has been discussed in relation to DSC, TGA and EGA analyses. The structure of the dehydrated hydrate has been identified using simultaneous scanning for structure via hot stage PXRD and low humidity PXRD of the solid obtained from the process of Example 4.

Hot stage powder X-ray diffraction was been performed using a Bruker D8 X-ray powder diffractometer fitted with Goebel mirrors and a Braun position sensitive detector. The sample of the solid obtained from the process of Example 4 was radiated using CuKα radiation. The sample of the solid obtained from the process of Example 4 was presented on a silicon wafer and heated to 100° C. at a rate of 0.1° C./sec using the Anton Paar heating stage fitted to the system. Following analysis at 100° C. the sample was allowed to cool to ambient and reanalysed five minutes and 2 hours after reaching ambient.

Low humidity PXRD was performed using an Ansyco humidity line model Sycos-H (AXS) using nitrogen as a carrier gas.

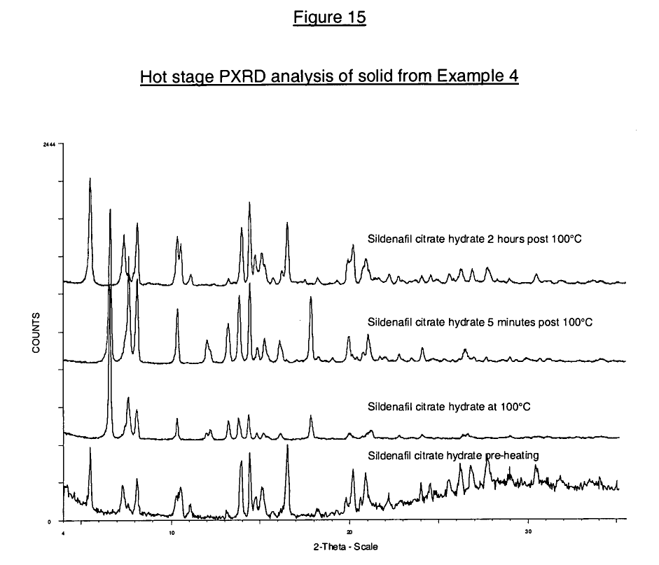

FIG. 15 illustrates the relative changes in the PXRD pattern associated with a sample of the solid obtained from the process of Example 4 when subjected to different heat treatments: RT; 100° C. (for about 20 minutes); 5 minutes after removal of 100° C. heat (i.e. 5 minutes cooling); and 2 hours after removal of 100° C. heat.

As illustrated in FIG. 15, hot stage analysis reveals that the characteristic peaks attributable to the higher hydrated form of sildenafil hemi-citrate are not present in the PXRD pattern for the sample analysed at 100° C. The peaks illustrated at 100° C. are attributable to a new “dehydrated hydrate”. The peak positions of this “dehydrated” hydrate may be shifted due to the thermal expansion of the atoms in the crystal lattice as will be appreciated by the skilled crystallographer. FIG. 15 further demonstrates that on cooling, after 2 hours the higher hydrated form is reproduced. These results indicate that on reaching 100° C. a new hygroscopic form has been produced which potentially due to its hygroscopicity reconverts to the hydrated form on returning to ambient temperatures.

Table 14 provides the peak positions of the dehydrated hydrate obtained from the solid obtained from the process of Example 4.

TABLE 14
Peak positions for dehydrated hydrate obtained from
the solid obtained from the process of Example 4

| Angle | Intensity |
|---|---|
| 2-Theta ° | % |
| 5.936 | 2.2 |
| 6.522 | 55.9 |
| 7.658 | 100 |
| 8.141 | 30.9 |
| 8.916 | 2.2 |
| 10.395 | 15.9 |
| 11.751 | 4.5 |
| 12.092 | 17.4 |
| 13.009 | 15.6 |
| 13.643 | 31.4 |
| 14.078 | 3.9 |
| 14.509 | 24.8 |
| 14.915 | 8.9 |
| 15.303 | 9.3 |
| 16.032 | 5.2 |
| 16.306 | 9.1 |
| 17.101 | 2 |
| 17.83 | 32.2 |
| 18.242 | 3.4 |
| 19.163 | 4.9 |
| 19.494 | 2.3 |
| 20.019 | 7.7 |
| 20.498 | 5.6 |
| 20.861 | 11.5 |
| 21.51 | 2.6 |
| 21.761 | 3 |
| 22.097 | 2.3 |
| 22.816 | 3.9 |
| 23.172 | 2.5 |
| 23.524 | 2.4 |
| 23.887 | 2.3 |
| 24.223 | 8.1 |
| 24.546 | 2.1 |
| 24.985 | 2.1 |
| 25.592 | 2.6 |
| 26.106 | 3.4 |
| 26.645 | 8.5 |
| 27.006 | 3.5 |
| 27.67 | 1.7 |
| 29.048 | 3.7 |
| 29.942 | 3.2 |
| 30.71 | 2 |
| 31.305 | 2.5 |
| 32.314 | 1.4 |
| 32.899 | 1.8 |
| 34.362 | 3 |

Thus the present invention provides a further solid having a PXRD pattern substantially as defined in Table 14 wherein said PXRD pattern is generated using CuKα₁ radiation (wavelength=1.5406 Å) and at 0% relative humidity when measured according to the method described hereinbefore.

TABLE 15
Peak positions for dehydrated hydrate of
Table 14 having at least 10% Intensity

| Angle | Intensity |
|---|---|
| 2-Theta ° | % |
| 6.522 | 55.9 |
| 7.658 | 100 |
| 8.141 | 30.9 |
| 10.395 | 15.9 |
| 12.092 | 17.4 |
| 13.009 | 15.6 |
| 13.643 | 31.4 |
| 14.509 | 24.8 |
| 17.83 | 32.2 |
| 20.861 | 11.5 |

Thus the present invention provides a solid having a PXRD pattern substantially as defined in Table 15 wherein said PXRD pattern is generated using CuKα₁ radiation (wavelength=1.5406 Å) and at 0% relative humidity when measured according to the method described hereinbefore.

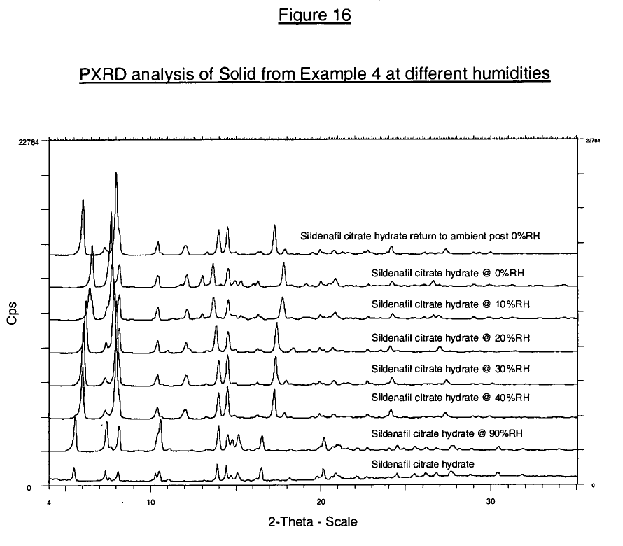

The traces in FIG. 16 indicate that on reduction of humidity (lower RH values) intermediate hydrated forms are present. At high humidity (90% RH) the trace is comparable to that of the sildenafil citrate higher hydrate illustrated in FIG. 11. On reducing the relative humidity (from 90%) in 10% steps no changes are observed until the humidity reaches 40% where the trace converts to a different hydrated form. Further stepwise reductions in humidity produce slight changes in the patterns with the major peak at an angle of 5.5° 2θ moving to higher angles. Without being bound to any particular theory we propose that this suggests a “contraction” is occurring within the crystal as water moves out of the lattice.

Thus the present invention additionally provides further hydrated forms of sildenafil citrate (1:0.75) at 40% RH, 20% RH and 10% RH having principal PXRD peaks as illustrated in FIG. 16.

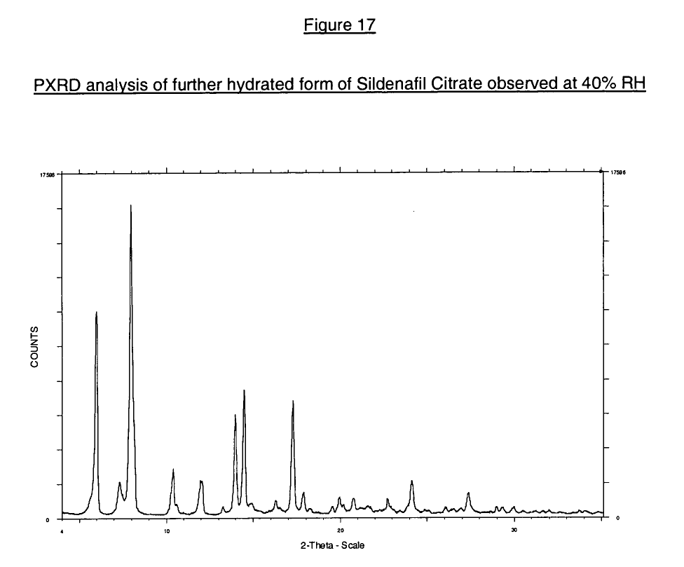

FIG. 17 illustrates the PXRD pattern of the hydrated form of sildenafil citrate observed at 40% RH.

According to a further embodiment the present invention provides a solid form having a powder X-ray diffraction (PXRD) pattern with main peaks substantially as defined in Table 16 wherein said PXRD pattern is generated using CuKα₁ radiation at a wavelength of 1.5406 Å and at ambient temperature and 40% RH.

TABLE 16

| Angle | Intensity |
|---|---|
| 2-Theta ° | % |
| 5.979 | 66 |
| 7.314 | 11.5 |
| 7.961 | 100 |
| 10.384 | 15.6 |
| 10.632 | 3.9 |
| 10.964 | 1.6 |
| 12.012 | 11.6 |
| 13.26 | 3.4 |
| 13.98 | 32.8 |
| 14.476 | 40.8 |
| 14.873 | 4.6 |
| 15.342 | 2 |
| 15.949 | 2 |
| 16.269 | 5.3 |
| 16.505 | 2.9 |
| 17.268 | 37.4 |
| 17.868 | 8 |
| 18.232 | 2.8 |
| 18.622 | 1.5 |
| 19.53 | 3.4 |
| 19.94 | 6.4 |
| 20.171 | 4 |
| 20.768 | 6 |
| 21.202 | 3 |
| 21.596 | 3.6 |
| 22.759 | 5.6 |
| 23.466 | 2.1 |
| 23.867 | 3.3 |
| 24.148 | 11.8 |
| 24.896 | 2.2 |
| 25.097 | 2.1 |
| 25.672 | 1.5 |
| 26.098 | 3.1 |
| 26.551 | 2.5 |
| 26.946 | 2.8 |
| 27.379 | 7.7 |
| 28.648 | 1.6 |
| 28.995 | 3.2 |
| 29.324 | 3 |
| 29.949 | 2.9 |
| 30.535 | 1.8 |
| 31.223 | 1.7 |
| 31.662 | 1.7 |
| 32.014 | 2.1 |
| 32.624 | 1.3 |
| 32.768 | 1.3 |
| 33.435 | 1.3 |
| 33.766 | 2 |
| 34.083 | 1.7 |
| 34.859 | 1.4 |

Table 17 illustrates the principal peaks of the solid form observed at 40% RH (i.e. those having an intensity of >10%). Thus according to a further still aspect the present invention provides a solid form having a powder X-ray diffraction (PXRD) pattern with main peaks substantially as defined in Table 17 and wherein said PXRD pattern is generated using CuKα₁ radiation (wavelength=1.5406 Å) at ambient temperature and 40% relative humidity.

TABLE 17

| Angle | Intensity |
|---|---|
| 2-Theta ° | % |
| 5.979 | 66 |
| 7.314 | 11.5 |
| 7.961 | 100 |
| 10.384 | 15.6 |
| 12.012 | 11.6 |
| 13.98 | 32.8 |
| 14.476 | 40.8 |
| 17.268 | 37.4 |
| 24.148 | 11.8 |

1. A solid form of sildenafil citrate wherein the ratio of sildenafil:citrate is about 1: about 0.5.

2. A hydrated solid form according to claim 1.

3. A hydrated solid form according to claim 2 wherein the amount of water present in the solid form is between about 4% and about 7% of dry weight of the solid.

4. A hydrated solid form of sildenafil hemi-citrate having a powder X-ray diffraction (PXRD) pattern with main peaks substantially as defined below:

| Angle (2-Theta) | Relative Intensity |
|---|---|
| 5.899 | 71.1 |
| 7.19 | 25.9 |
| 7.905 | 100 |
| 10.523 | 14.4 |
| 11.905 | 21.2 |
| 13.25 | 16.9 |
| 13.893 | 83.7 |
| 14.371 | 50.9 |
| 15.104 | 15.1 |
| 15.373 | 17.9 |
| 15.948 | 18.1 |
| 16.431 | 20.1 |
| 17.221 | 64 |
| 17.812 | 25 |
| 18.668 | 14.2 |
| 19.492 | 15.8 |
| 20.38 | 18 |
| 20.708 | 32.6 |
| 21.318 | 23.5 |
| 21.5 | 22.4 |
| 22.256 | 17.8 |
| 22.493 | 22 |
| 22.86 | 23.2 |
| 23.415 | 21.3 |
| 23.86 | 24.7 |
| 24.145 | 45.1 |
| 25.075 | 33.6 |
| 25.565 | 16 |
| 26.086 | 23.9 |
| 26.506 | 36.8 |
| 27.356 | 27.2 |
| 28.24 | 15.9 |
| 29.02 | 15.3 |
| 29.207 | 17 |
| 29.799 | 16.5 |
| 29.926 | 19 |
| 30.361 | 13 |
| 31.01 | 13.2 |
| 31.891 | 14.6 |
| 32.556 | 15.3 |
| 33.073 | 12.5 |
| 33.4 | 16.2 |
| 33.768 | 12.8 |
| 34.219 | 13.7 |
| 34.38 | 13.3 |
| 34.752 | 13.9 |
| 35.332 | 14.1 |
| 36.115 | 13.3 |
| 37.122 | 15.1 |
| 37.514 | 15.7 |
| 38.69 | 13.3 |
| 39.009 | 12.8 |

wherein said PXRD pattern is generated using CuKα₁ radiation at a wavelength of 1.5406 Å and at ambient temperature and humidity.

5. A hydrated solid form of sildenafil hemi-citrate having a powder X-ray diffraction (PXRD) pattern with main peaks substantially as defined below:

| Angle (2-Theta) | Relative Intensity |
|---|---|
| 5.899 | 71.1 |
| 7.905 | 100 |
| 13.893 | 83.7 |
| 14.371 | 50.9 |
| 17.221 | 64 |
| 20.708 | 32.6 |
| 24.145 | 45.1 |
| 25.075 | 33.6 |
| 26.506 | 36.8 |
| 27.356 | 27.2 |

wherein said PXRD pattern is generated using CuKα₁ radiation at a wavelength of 1.5406 Å and at ambient temperature and humidity.

6. A hydrated solid form of sildenafil citrate according to claim 2 wherein the hydrated solid form is a hemi-citrate having a PXRD pattern as defined in claims 4 or 5.

7. A hydrated solid form according to claim 2 wherein the amount of water present in the solid form is between about 12% and about 14% of dry weight of the solid.

8. A hydrated solid form of sildenafil hemi-citrate having a powder X-ray diffraction (PXRD) pattern with main peaks substantially as defined below:

| Angle (2-Theta) | Relative Intensity |
|---|---|
| 5.525 | 48.1 |
| 7.387 | 22.3 |
| 7.671 | 10 |
| 10.561 | 35.9 |
| 11.125 | 11.1 |
| 13.524 | 10.8 |
| 14.004 | 50.8 |
| 14.764 | 25.6 |
| 15.162 | 38.4 |
| 15.298 | 30.4 |
| 15.787 | 11.5 |
| 16.56 | 100 |
| 19.329 | 15.2 |
| 20.214 | 43.7 |
| 20.777 | 18.5 |
| 20.943 | 28.1 |
| 21.112 | 28.6 |
| 21.457 | 18.5 |
| 21.605 | 19.8 |
| 22.25 | 15.1 |
| 23.117 | 10.5 |
| 23.727 | 15.1 |
| 24.096 | 23.9 |
| 24.576 | 20.3 |
| 24.998 | 17 |
| 25.587 | 21.1 |
| 25.845 | 19.3 |
| 26.279 | 21.2 |
| 26.885 | 19 |
| 27.16 | 11.9 |
| 27.44 | 13.5 |
| 27.68 | 46.4 |
| 27.92 | 26.2 |
| 28.295 | 17.4 |
| 29.085 | 10.3 |
| 30.464 | 19.7 |
| 30.875 | 11.1 |
| 31.046 | 11.2 |
| 31.959 | 13.8 |
| 33.4 | 11.1 |
| 33.6 | 13.1 |
| 34.087 | 10.8 |
| 35.094 | 11.7 |
| 36.4 | 10.2 |
| 36.579 | 11.3 |
| 37.339 | 10.6 |
| 38.624 | 10.2 |
| 39.148 | 18.4 |
| 39.608 | 12.2 |

wherein said PXRD pattern is generated using CuKα₁ radiation at a wavelength of 1.5406 Å and at ambient temperature and from 40 to 60% RH.

9. A hydrated solid form of sildenafil hemi-citrate having a powder X-ray diffraction (PXRD) pattern with main peaks substantially as defined below:

| Angle (2-Theta) | Relative Intensity |
|---|---|
| 5.525 | 48.1 |
| 10.561 | 35.9 |
| 14.004 | 50.8 |
| 15.162 | 38.4 |
| 15.298 | 30.4 |
| 16.56 | 100 |
| 20.214 | 43.7 |
| 20.943 | 28.1 |
| 21.112 | 28.6 |
| 27.68 | 46.4 |

wherein said PXRD pattern is generated using CuKα₁ radiation (wavelength=1.5406 Å) at ambient temperature and 40 to 60% relative humidity.

10. A hydrated solid form of sildenafil citrate according to claim 2 wherein the hydrated solid form is a hemi-citrate having a PXRD pattern as defined in claim 8 or 9.

11. A solid form of sildenafil hemi-citrate according to claim 1 having a powder X-ray diffraction (PXRD) pattern with main peaks substantially as defined below:

| Angle (2-Theta) | Relative Intensity |
|---|---|
| 5.188 | 6.6 |
| 6.473 | 61.6 |
| 7.624 | 100 |
| 10.382 | 5.4 |
| 11.702 | 8.7 |
| 12.046 | 14.9 |
| 12.965 | 15.4 |
| 13.621 | 35.3 |
| 14.263 | 7.9 |
| 14.902 | 10.1 |
| 15.28 | 12 |
| 15.986 | 17.5 |
| 16.239 | 18.2 |
| 17.032 | 7.7 |
| 17.795 | 21.9 |
| 18.139 | 6.5 |
| 19.126 | 6.7 |
| 20.452 | 13.7 |
| 20.897 | 16.1 |
| 21.483 | 7 |
| 23.352 | 8.9 |
| 24.182 | 8.6 |
| 24.926 | 8.7 |
| 25.526 | 9.1 |
| 26.635 | 8.6 |
| 26.98 | 6.7 |

wherein said PXRD pattern is generated using CuKα₁ radiation at a wavelength of 1.5406 Å under vacuum and at ambient temperature.

12. A solid material as defined in any of FIGS. 10 to 17 herein.

13. A hydrated solid form of sildenafil citrate according to claim 3 wherein the hydrated solid form is a hemi-citrate having a PXRD pattern as defined in claims 4 or 5.

14. A hydrated solid form of sildenafil citrate according to claim 7 wherein the hydrated solid form is a hemi-citrate having a PXRD pattern as defined in claim 8 or 9.
